(19) United States
(12) Patent Application Publication
(10) Pub. No.: US 2020/0352411 A1
(43) Pub. Date: Nov. 12, 2020

(54) RECOMMENDED OPERATION PRESENTATION SYSTEM, RECOMMENDED OPERATION PRESENTATION CONTROL DEVICE, AND RECOMMENDED OPERATION PRESENTATION CONTROL METHOD

(71) Applicant: OLYMPUS CORPORATION, Tokyo (JP)

(72) Inventors: Ryo TOJO, Hachioji-shi (JP); Hiromasa FUJITA, Hachioji-shi (JP)

(73) Assignee: OLYMPUS CORPORATION, Tokyo (JP)

(21) Appl. No.: 16/909,050

(22) Filed: Jun. 23, 2020

Related U.S. Application Data
(63) Continuation of application No. PCT/JP2017/046421, filed on Dec. 25, 2017.

(51) Int. Cl. A61B 1/00; A61B 1/005; G06K 9/62

(52) U.S. Cl. CPC A61B 1/00006; A61B 1/00039; G06K 9/6209; A61B 1/00055; A61B 1/0051

(57) ABSTRACT

A recommended operation presentation system includes an insertion instrument including an insertion section to be inserted into an insertion object and an insertion section shape detector configured to detect shape information of the insertion section. The system also includes an attention state/operation control information arithmetic operator configured to calculate operation control information changing in association with an operation of the insertion instrument, based on the shape information and target state information, and a recommended operation arithmetic operator configured to calculate recommended operation information based on the operation control information.

### CROSS-REFERENCE TO RELATED APPLICATIONS

This application is a Continuation Application of PCT Application No. PCT/JP2017/046421, filed Dec. 25, 2017, the entire contents of which are incorporated herein by reference.

### BACKGROUND OF THE INVENTION

### 1. Field of the Invention

The present invention relates to a recommended operation presentation system configured to output a recommended operation of an insertion instrument, a recommended operation presentation control device, and a recommended operation presentation control method.

### 2. Description of the Related Art

For example, Jpn. Pat. Appln. KOKAI Publication No. 2004-358095 discloses an endoscope insertion analysis apparatus configured to display shape information and operation instruction information in accordance with an insertion operation when an insertion section forms a loop in an object during endoscopic examination or when an observation region of the object is extended during endoscopic examination.

### BRIEF SUMMARY OF THE INVENTION

A recommended operation presentation system according to the present invention includes: an insertion instrument including an insertion section to be inserted into an insertion object; an insertion section shape detector configured to detect a shape of at least part of the insertion section as shape information; an attention state/operation control information arithmetic operator configured to calculate operation control information that is information changing in association with an operation of the insertion instrument, based on the shape information and target state information, the target state information being set based on the shape information and preliminarily prepared recommendation operation reference data; a recommended operation arithmetic operator configured to calculate recommended operation information including a recommended operation direction that is an operation direction of a recommended insertion operation and a recommended operation amount that is an operation amount of the recommended insertion operation, based on at least the operation control information; and an output information processor configured to output information including the recommended operation information.

A recommended operation presentation control device according to the present invention includes: an attention state/operation control information arithmetic operator configured to calculate operation control information that is information changing in association with an operation of an insertion instrument including an insertion section configured to be inserted into an insertion object, based on shape information and target state information, the shape information relating to a shape of at least part of the insertion section, the target state information being set based on the shape information and preliminarily prepared recommendation operation reference data; a recommended operation arithmetic operator configured to calculate recommended operation information including a recommended operation direction that is an insertion direction of a recommended insertion operation and a recommended operation amount that is an operation amount of the recommended insertion operation, based on the at least operation control information; and an output information processor configured to output information including the recommended operation information.

A recommended operation presentation control method according to the present invention includes: calculating operation control information that is information changing in association with an operation of an insertion instrument including an insertion section configured to be inserted into an insertion object, based on shape information and target state information, the shape information relating to a shape of at least part of the insertion section, the target state information being set based on the shape information and preliminarily prepared recommendation operation reference data; calculating recommended operation information including a recommended operation direction that is an insertion direction of a recommended insertion operation and a recommended operation amount that is an operation amount of the recommended insertion operation, based on at least the operation control information; and outputting information including the recommended operation information.

Advantages of the invention will be set forth in the description that follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram schematically showing an example of an endoscopic system.

FIG. 2 is a block diagram showing the example of the endoscopic system.

FIG. 3A is a diagram showing an example of an α-loop of an insertion section.

FIG. 3B is a diagram showing an example of an α-loop of the insertion section.

FIG. 3C is a diagram showing an example of an α-loop of the insertion section.

FIG. 3D is a diagram showing an example of an α-loop of the insertion section.

FIG. 3E is a diagram showing an example of an N-loop of the insertion section.

FIG. 3F is a diagram showing an example of an N-loop of the insertion section.

FIG. 4A is a flowchart showing an example of the processing of recommended operation information by a control device of the endoscope system.

FIG. 4B is a flowchart showing an example of the processing of recommended operation information by the control device of the endoscope system.

FIG. 5 is a diagram showing an example of recommended operation reference data.

FIG. 6 is a diagram showing another example of the recommended operation reference data.

FIG. 7A illustrates an example of the state of the insertion section relating to N-loop release.

FIG. 7B illustrates an example of the state of the insertion section relating to N-loop release.

FIG. 7C is a diagram for explaining an operation amount relating to a clockwise twisting operation.

FIG. 7D is a diagram showing an example of the state of the insertion section relating to N-loop release.

FIG. 7E is a diagram showing an example of the state of the insertion section relating to N-loop release.

FIG. 8A is a diagram showing an example of the state of an insertion section relating to a loop release.

FIG. 8B is a diagram showing an example of the state of an insertion section relating to a loop release.

FIG. 8C is a diagram showing an example of the state of an insertion section relating to a loop release.

FIG. 9 is a diagram showing an example of a display screen of a display device.

FIG. 10A is a diagram showing an example of the display of recommended operation information.

FIG. 10B is a diagram showing an example of the display of recommended operation information.

FIG. 100 is a diagram showing an example of the display of recommended operation information.

FIG. 11A is a diagram showing an example of the display of recommended operation information.

FIG. 11B is a diagram showing an example of the display of recommended operation information.

FIG. 11C is a diagram showing an example of the display of recommended operation information.

FIG. 12A is a diagram showing an example of the display of recommended operation information.

FIG. 12B is a diagram showing an example of the display of recommended operation information.

FIG. 12C is a diagram showing an example of the display of recommended operation information.

FIG. 13 is a diagram showing an example of recommended operation information superimposed on an endoscope image.

FIG. 14 is a diagram showing an example of the display of recommended operation information.

FIG. 15 is a diagram showing an example of loop occurrence time and display time of attention state information/recommended operation information.

FIG. 16 is a diagram showing an example of the loop occurrence time and the display time of attention state information/recommended operation information.

FIG. 17 is a diagram showing an example of an endoscope image including a lumen.

FIG. 18 is a block diagram showing an example of an endoscope system according to a modification 1.

FIG. 19A is a flowchart showing an example of the processing of recommended operation information according to the modification 1.

FIG. 19B is a flowchart showing the example of the processing of recommended operation information according to the modification 1.

FIG. 20 is a block diagram showing an example of an endoscope system according to a modification 2.

FIG. 21 is a diagram showing an example of a display screen of a display according to the modification 2.

FIG. 22 is a diagram schematically showing an example of an endoscope system according to a modification 3.

### DETAILED DESCRIPTION OF THE INVENTION

An embodiment of the present invention will be described with reference to the drawings.

Citing an example of a large intestine flexible endoscope for medical treatment (large intestine endoscope) as an insertion instrument, a recommended operation presentation system according to the embodiment of the present invention will be described with reference to an example of an insertion system including the endoscope. The insertion instrument is not limited to a medical endoscope, and it suffices that the insertion instrument is to be inserted into an object, such as a catheter, a treatment device, an industrial endoscope, etc., and at least a part of which has flexibility. The object is not limited to humans, but may be an insertion object including an animal, an engine, a structure including piping, or the like.

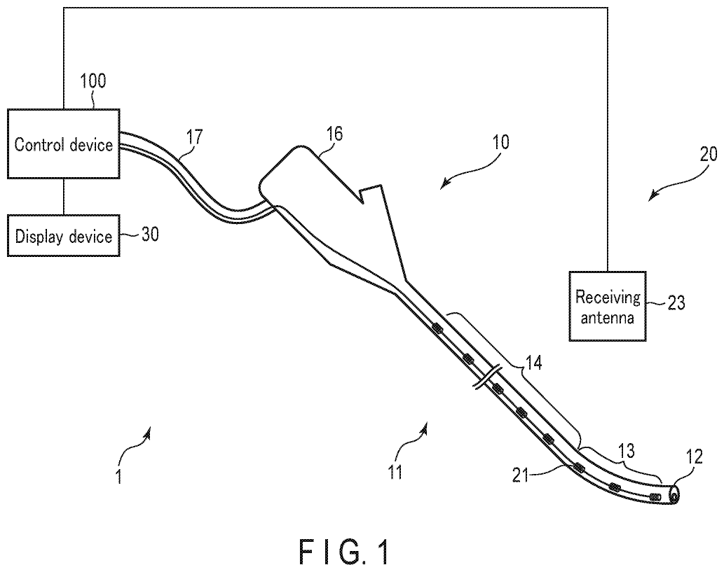

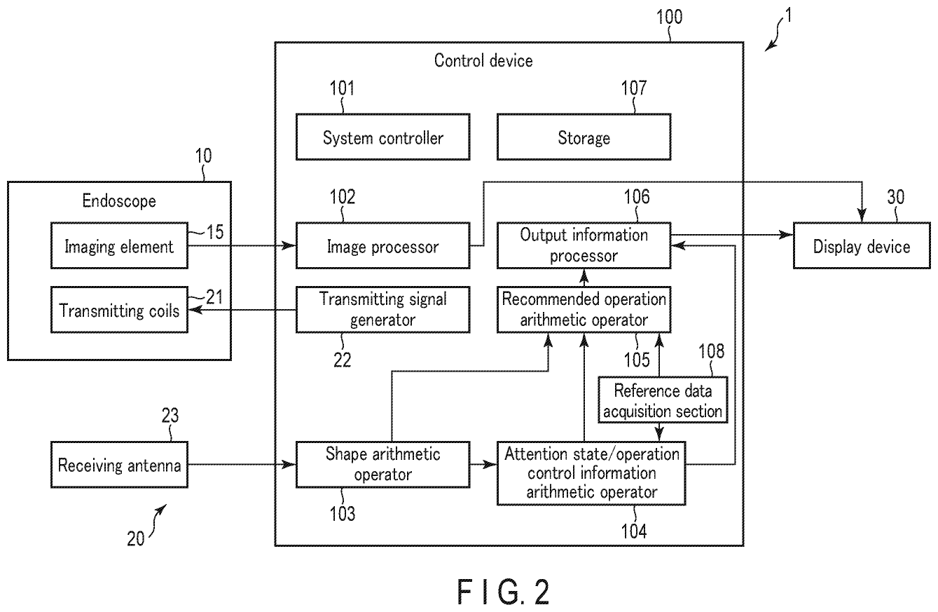

FIG. 1 is a diagram schematically showing an example of an endoscope system 1. FIG. 2 is a block diagram showing an example of the endoscope system 1. The endoscope system 1 includes an endoscope 10, a position detection device 20, a display device 30, and a control device 100.

[Endoscope]

The endoscope 10 includes an insertion section 11 at the distal end, an operation section 16 on the proximal end side of the insertion section 11, and a cable 17. The insertion section 11 has a distal hard section 12, an operationally bendable section 13 on its proximal end side, and a passive bendable section 14 on the proximal end side of the operationally bendable section 13. The distal hard section 12 incorporates an illuminating optical system, an observation optical system, an imaging element 15, and the like. The operationally bendable section 13 is a portion within, for example, a range of approximately 15 cm from the proximal end of the distal hard section 12 in the insertion section 11. The operationally bendable section 13 can be bended in a desired direction by operating an operation knob (not shown) (e.g., a rotation operation) of the control section 16. The passive bendable section 14 is freely bendable by an external force or the like received from an object. The endoscope 10 is connected to the control device 100 via the cable 17.

The insertion section 11, for example, the operationally bendable section 13 and the passive bendable section 14, is provided with transmitting coils 21, which constitute part of the position detection device 20. The transmitting coils 21 are arranged to be spaced apart from each other along the longitudinal direction of the insertion section 11. In FIG. 1, three transmitting coils 21 are arranged in different positions in the longitudinal direction of the operationally bendable section 13. In addition, 10 transmitting coils 21 (part of which is omitted in FIG. 1) are arranged at different positions in the passive bendable section 14 in the longitudinal direction of the passive bendable section 14.

[Position Detection Device]

In the present embodiment, the position detection device 20 is of a magnetic type. The position detection device 20 includes transmitting coils 21, a transmitting signal generator 22, and a receiving antenna 23. The transmitting coils 21 are incorporated in the insertion section 11 of the endoscope 10 as described above. The transmitting signal generator 22 is incorporated in, for example, the control device 100. Of course, the configuration of the transmitting signal generator 22 is not limited thereto, and it may be separated from the control device 100. The receiving antenna 23 is separated from the endoscope 10 and the control device 100. It may be configured that the receiving antenna 23 is replaced with a transmitting antenna and the insertion section 11 of the endoscope 10 is provided with receiving coils, such that transmission and reception are inversed.

The transmitting signal generator 22 generates signals for causing the transmitting coils 21 to generate magnetic fields, for example, currents of a sinusoidal wave. The generated signals are sequentially output to the transmitting coils 21 in the order determined for the transmitting coils 21, for example, from the distal end side of the insertion section 11. The transmitting coil 21 generates a magnetic field by a current flowing from the transmitting signal generator 22. In FIG. 1, only a single piece of wiring connected to the transmitting coils 21 is depicted, but two pieces of wiring are connected to a transmitting coil 21.

The receiving antenna 23 comprises receiving coils (not shown). The receiving antenna 23 detects magnetic fields signal generated by the transmitting coils 21. The receiving antenna 23 is located and positioned with respect to a room or a bed so as not to move. The positions of the transmitting coils 21 are calculated by a shape arithmetic operator 103, which is described later, in the control device 100, based on the strength of the magnetic fields detected by the receiving antenna 23. In FIG. 1, the receiving antenna 23 and the control device 100 are wiredly connected, but they may be wirelessly connected.

The position detection device 20 only needs to detect a bending state of the insertion section 11 in order to function as an insertion section shape detector configured to detect a shape of at least part of the insertion section 11, so may be of any type other than the magnetic type. The position detection device 20 may be composed of, for example, any one of a sensing (magnetic sensor) using magnetism, a sensing using an ultrasonic wave (ultrasonic sensor), a sensing using a loss of light guided by an optical fiber (optical fiber sensor), a sensing using distortion (a distortion sensor) using distortion, and a sensing using an x-ray absorption material, or a combination thereof. Here, it is referred to as the “position detection device”, but it may be a device having the same function as that of a device referred to as a “shape detector”.

[Control Device]

The control device 100 includes a system controller 101, an image processor 102, a shape arithmetic operator 103, an attention state/operation control information arithmetic operator 104, a recommended operation arithmetic operator 105, an output information processor 106, and a reference data acquisition section 108. These sections are composed of a processor including one or more integrated circuits, such as a CPU. The control device 100 also includes a storage 107. The storage 107 is a storage such as a semiconductor memory.

The above-mentioned sections of the control device 100, especially the shape arithmetic operator 103, attention state/operation control information arithmetic operator 104, recommended operation arithmetic operator 105, and reference data acquisition section 108 may be included in a control device separate from the control device 100. For example, the shape arithmetic operator 103, attention state/operation control information arithmetic operator 104, recommended operation arithmetic operator 105, and reference data acquisition section 108 may be included in a control device other than an endoscope video image processor including the image processor 102. Alternatively, the shape arithmetic operator 103, attention state/operation control information arithmetic operator 104, recommended operation arithmetic operator 105, and reference data acquisition section 108 may be included in other control devices, respectively. Namely, the above-described sections of the control device 100, especially the processors or hardware circuits functioning as the shape arithmetic operator 103, the attention state/operation control information arithmetic operator 104, the recommended operation arithmetic operator 105, and reference data acquisition section 108 may be included in a single housing or may be included in several housings, provided that these sections can perform their respective functions.

[System Controller]

The system controller 101 controls the operations of various devices of the endoscope system 1, which are connected to the control device 100. The system controller 101 controls a light source device (not shown) connected to the control device 100 in order to supply illuminating light to the illuminating optical system of the distal hard section 12 of the endoscope 10, for example.

[Image Processor]

The image processor 102 converts an electric signal into which light from an object has been converted by the imaging element 15 at the distal hard section 12 of the endoscope 10 into a video signal to generate an endoscope image based on an object image, and causing the display device 30 to display the endoscope image.

[Shape Arithmetic Operator]

The shape arithmetic operator 103 calculates a position and a direction vector of each transmitting coil 21 as positional information of each transmitting coil 21 based on the intensity information of a magnetic field input from the receiving antenna 23. Since the plurality of pieces of positional information obtained by the transmitting coils 21 represents the shape of the insertion section 11, the plurality of pieces of positional information is referred to as shape information in the present embodiment. The positional information of the transmitting coils 21 may be interpolated by spline processing or the like, if necessary. The shape information is output to the attention state/operation control information arithmetic operator 104 and the recommended operation arithmetic operator 105. As described above, in the present embodiment, the shape arithmetic operator 103 functions, together with the position detection device 20, as an insertion section shape detector configured to detect a shape of at least part of the insertion section 11, as shape information.

[Attention State/Operation Control Information Arithmetic Operator]

The attention state/operation control information arithmetic operator 104 calculates attention state information and operation control information from the shape information. The attention state information and the operation control information is output to the recommended operation arithmetic operator 105 and the output information processor 106. The attention state/operation control information arithmetic operator 104 may be divided into an attention state arithmetic operator configured to calculate attention state information and an operation control information arithmetic operator configured to calculate operation control information.

The attention state information is information for determining whether or not recommended operation information to be described later is necessary or information for determining the recommended operation information, which is information relating to information relating to a predetermined state of the insertion section 11, for example, a characteristic state that is preliminarily assumed to occur when the insert section 11 is inserted into an object. The attention state information is, for example, information on the presence or absence of a loop of the insertion section 11 of the endoscope 10, the type and size of the loop (such as a loop area, a circumference, a width, etc.), an insertion length or a movement amount of the distal end of the insertion section, a change of the insertion section distal end with respect to a change in the insertion length or a proportion of the movement amount of the insertion section distal end with respect to the insertion amount of insertion, etc. The insertion length is, for example, a length of a portion of the insertion section 11 inserted into an object. The change of the insertion section distal end with respect to the change in the insertion length is, for example, information for determining whether or not the distal end of the insertion section 11 proceeds with respect to the insertion operation, i.e., whether the insertion is not prevented.

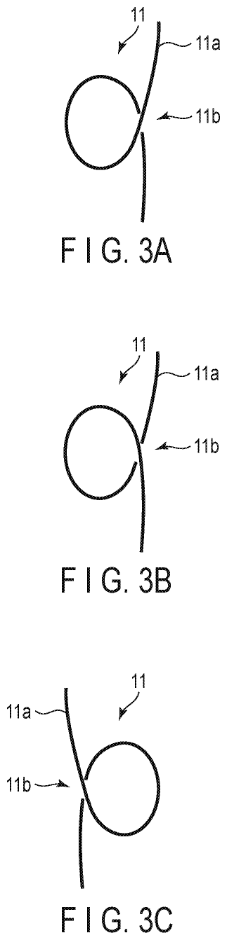

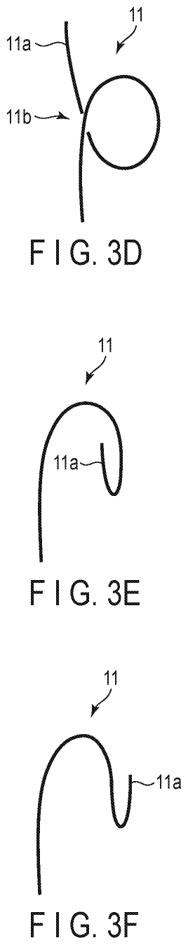

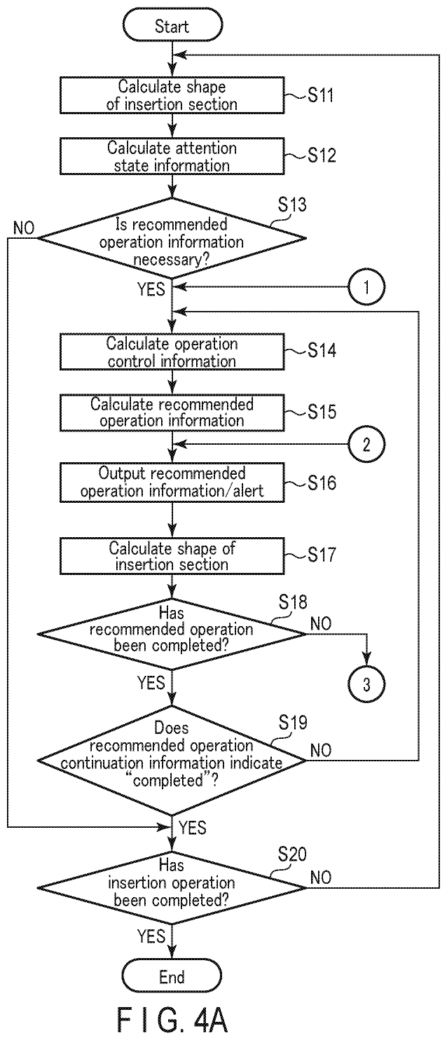

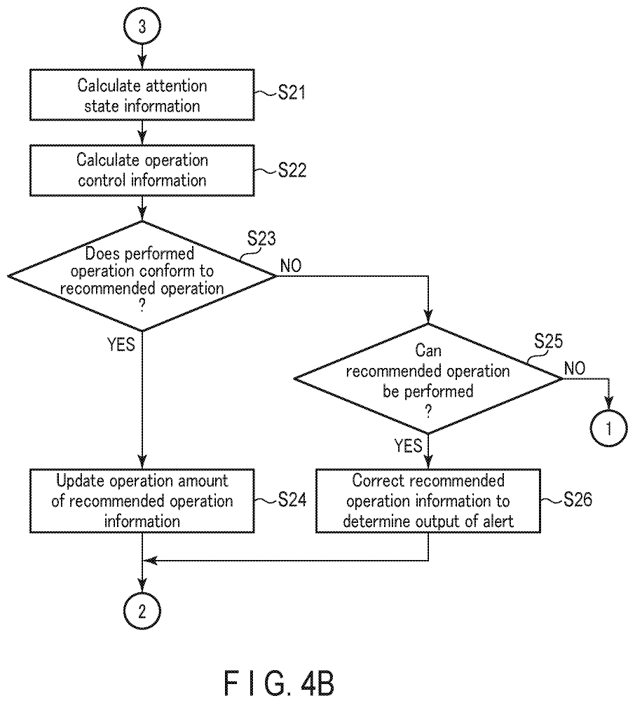

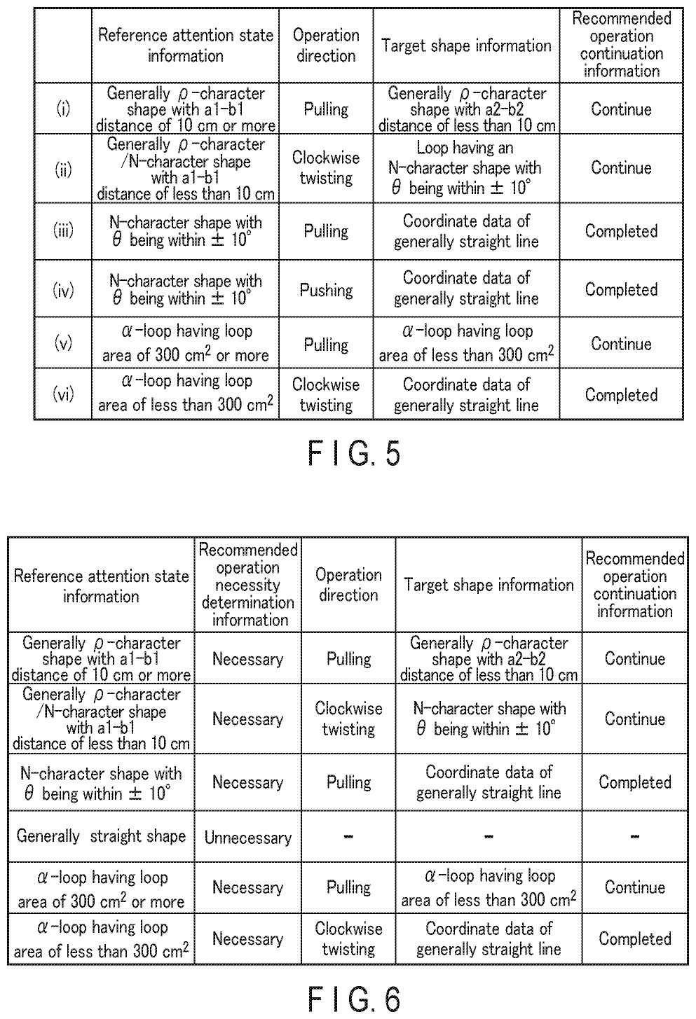

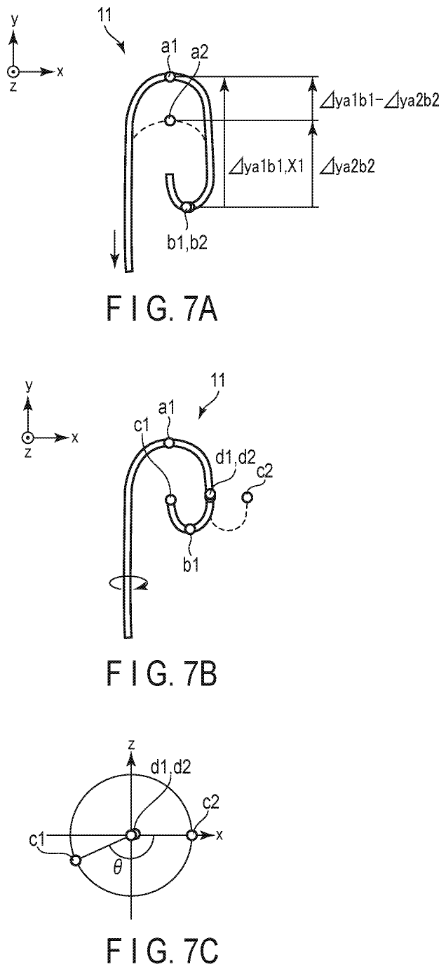

FIGS. 3A to 3F show some examples of a loop of the insertion section 11. In FIGS. 3A to 3F, the upper side of the paper sheet is the distal end side of the insertion section 11, and the lower side of the paper sheet is the proximal end side (hand side) of the insertion section 11. FIG. 3A shows an α-loop in which the side of an insertion section distal end 11a is present on the upper side at an intersecting portion 11b of the insertion section 11. FIG. 3B shows an α-loop in which the side of the insertion section distal end 11a is present on the lower side at the intersecting portion 11b (referred to as a “top-bottom reversed α-loop” where the shape of the loop is similar to “α” and the top of the insertion section distal end 11a is reversed with respect to the proximal end side of the insertion section 11). FIG. 3C shows a reversed α-loop in which the side of the insertion section distal end 11a is present on the upper side at the intersecting portion 11b (referred to as a “top-bottom reversed and left-right reversed α-loop” where the left and right of the loop is reversed in addition to the reverse of the top and left). FIG. 3D shows a reversed α-loop in which the side of the insertion section distal end 11a is present on the lower side at the intersecting portion 11b. FIG. 3E shows a loop in which the side of the insertion section distal end 11a of an N-loop is present inside the loop, which is referred to as a “generally ρ-character shape”, (that is, the loop having a shape similar to a Greek alphabet lower-case rho “ρ”). FIG. 3F shows an N-loop having an N-character shape in which the side of the insertion section distal end 11a is present outside the loop. Strictly speaking, the generally ρ-character shape and N-character shape are not loops, but these are generally treated as loops, which are each assumed as one of N-loops in the present application. These are loops that may be formed in the sigmoid colon when the endoscope 10 is inserted into the large intestine. For example, in the case where the object is the large intestine, the α-loop of FIG. 3A and the reversed α-loop of FIG. 3C are loops in which the side of the insertion section distal end 11a lies on ventral side at the intersecting portion 11b of the insertion section 11, and the α-loop of FIG. 3B and the reversed α-loop of FIG. 3D are loops in which the side of the insertion section distal end 11a lies on dorsal side at the intersecting portion 11b. When such a loop is formed, the operator cannot easily insert the insertion section 11 into the intestinal tract. Therefore, in the present embodiment, such a loop is deemed an attention state, and attention state information is calculated by the attention state/operation control information arithmetic operator 104.

The operation control information is information relating to an operation of the endoscope 10 and is information directly or indirectly changed in association with the operator's operation on the endoscope 10. The operator's operation is mainly a bending operation, a twisting operation, and an insertion/extraction operation. As described above, the bending operation is an operation where the operator bends an operationally bendable section 13 by operating the control section 16. The twisting operation is an operation where the operator twists the passive bendable section 14 to the left side or the right side from its hand side. The twisting operation is also referred to as a rotation operation. The insertion/extraction operation is an operation where the operator pushes the passive bendable section 14 from its hand side (pushing operation) or an operation where the operator extracts the passive bendable section 14 from its hand side (pulling operation). In addition, the operation may be, for example, air supply, water supply, and suction through the endoscope 10 using an operation button (not shown) of the control section 16, a change in hardness of the insertion section 11, and hand compression for suppressing a change in shape of the insertion section 11 by pressing, for example, the abdomen of an object by hand.

For example, if a pulling operation is performed from a state where an α-loop as shown in FIGS. 3A to 3D is formed, the area and the circumference of the α-loop are reduced. The area and the circumference of the α-loop are operation control information that changes directly in association with an operator's operation.

Alternatively, a target shape of the insertion section 11 is set in order to determine a recommended operation. The difference between the target shape and a current shape (e.g., a total of differences in coordinates between the target shape of the insertion section 11 and the current shape of the insertion section 11) is reduced by performing an appropriate operation. Therefore, the difference is due to operation control information that changes indirectly in association with an operator's operation. As described above, the operation control information can be obtained by indirectly detecting information relating to the operation even if it is difficult to directly detect the information.

As the operation control information, for example, the attention state/operation control information arithmetic operator 104 may calculate a bending operation direction and a bending amount from the change in the shape information. In a case where a 1-axis transmitting coil 21 is provided in the insertion section 11, the bending operation direction cannot be detected; however, for example, providing an additional coil near the 1-axis coil in a different direction to constitute a 2-axis coil allows detecting the bending operation direction. In the case of the 1-axis coil, since the rotation of the coil around the axis of the coil cannot be detected, it is difficult to detect a twisting operation; however, replacing the 1-axis coil with the 2-axis coil allows detecting the twisting operation. The insertion/extraction operation can be calculated from a positional relationship between the position of the anus and coordinates of the insertion section 11 by storing the position of the anus at the start of insertion in the storage 107.

The operation control information may be information based on a detection result obtained from sensors provided on the endoscope 10 to detect various operator's operations on the endoscope 10. For example, in a case where the operationally bendable section 13 is bent by the rotation of an operation knob (not shown) of the control section 16, a rotary encoder configured to detect a rotation amount of the operation knob is provided on the operation section 16. This allows detection of the operation control information relating to bending operations.

[Recommended Operation Arithmetic Operator]

The recommended operation arithmetic operator 105 calculates recommended operation information that is information relating to a operation recommended for smoothly proceeding with insertion of the insertion section 11 based on a result of the calculations at the shape arithmetic operator 103 and the attention state/operation control information arithmetic operator 104, i.e., the shape information, attention state information, and operation control information. The recommended operation information includes, for example, a recommended operation direction that is an operation direction of a recommended insertion operation and a recommended operation amount that is an operation amount of the recommended insertion operation. The recommended operation information is output to the output information processor 106.

[Output Information Processor]

The output information processor 106 outputs information including recommended operation information. For example, the output information processor 106 performs image processing for displaying recommended operation information on the display device 30 and causes the display device 30 to display the recommended operation information. Besides the recommended operation information, the output information processor 106 may perform image processing for displaying information, such as shape information and attention state information, on the display device 30.

[Storage]

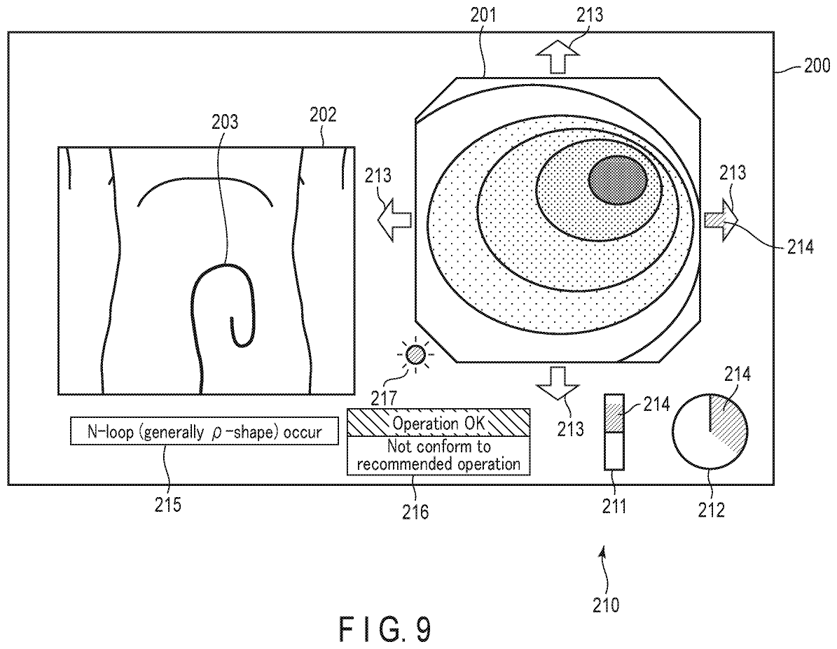

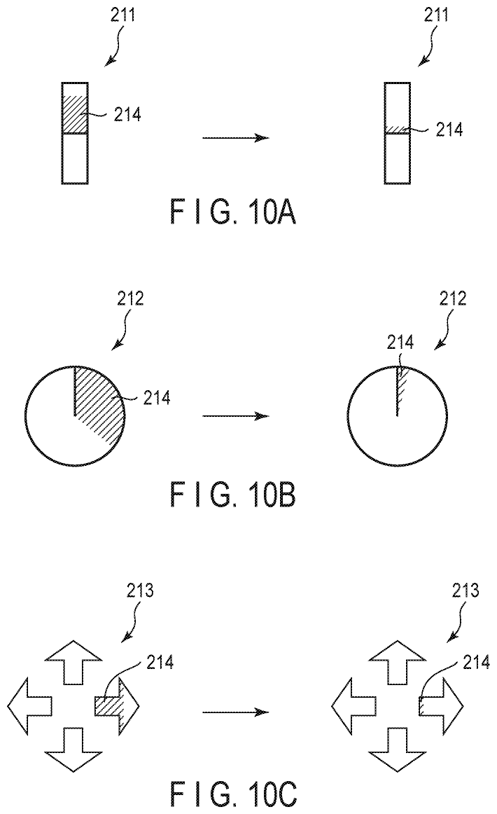

The storage 107 stores various programs and data necessary for operations of various devices connected to the control device 100 and operations of various components of the control device 100. The storage 107 stores recommended operation reference data relating to an insertion of the endoscope 10 into the large intestine as shown in FIG. 5 or 6, for example. The recommended operation reference data includes preliminarily patterned operation directions and target state information for releasing a loop when the above-mentioned attention state, such as an N-loop and an α-loop, has occurred. The recommended operation reference data may be in a format that is stored in and read out from an external storage device other than the storage 107. The storage 107 may store various pieces of data generated during the insertion.

FIG. 5 shows, as an example of the recommended operation reference data, reference attention state information preliminarily set so as to correspond to the attention state information, an operation direction, target shape information (target state information), and recommended operation continuation information preliminarily set so as to correspond to the reference attention state information. FIG. 6 shows, as other examples of the recommended operation reference data, reference attention state information preliminarily set so as to correspond to attention state information, and the recommended operation necessity determination information, operation direction, target shape information (target state information), and recommended operation continuation information preliminarily set so as to correspond to the reference attention state information. The reference attention state information is information relating to a predetermined state of the insertion section 11 and corresponds to the attention state information calculated by the attention state/operation control information arithmetic operator 104. The target state information also corresponds to the attention state information. As described above, the reference attention state information and the target state information corresponds to the attention state information and is, for example, information such as the presence or absence of a loop of the insertion section 11 of the endoscope 10, the type and size of the loop, the insertion length, and a change of the insertion distal end with respect to a change in insertion length. The recommended operation reference data is referred to in the calculation by the attention state/operation control information arithmetic operator 104 and the recommended operation arithmetic operator 105. Of course, the recommended operation reference data is not limited thereto, and various pieces of recommended operation reference data are used in accordance with an object.

For example, items (i), (ii), (iii), and (iv) of the recommended operation reference data are recommended operation reference data for release of an N-loop release (straightening of an N-loop) formed in the insert section 11. In the recommended operation presentation system according to the present embodiment, the operation control information and the recommended operation information is calculated so as to correspond to the reference attention state information calculated by the attention state/operation control information arithmetic operator 104 in the order of the items (i), (ii), and (iii), or in the order of the items (ii) and (iii), or with reference to reference data of (iii). The reference data of the item (iv) is also referred to as necessary. Similarly, the items (v) and (vi) of the recommended operation reference data are the recommended operation reference data for releasing an α-loop (straightening of an α-loop) formed by the insertion section 11. In the recommended operation presentation system according to the present embodiment, the operation control information and the recommended operation information are calculated so as to correspond to the reference attention state information calculated by the attention state/operation control information arithmetic operator 104 in the order of the items (v) and (vi), or with reference to the reference data of the item (vi) as described later.

[Reference Data Acquisition Section]

The reference data acquisition section 108 acquires the recommended operation reference data stored in the storage 107 or an external storage device other than the storage 107. The attention state/operation control information arithmetic operator 104 and the recommended operation arithmetic operator 105 perform a calculation of the attention state information, a calculation of the operation control information, and a calculation of the recommended operation information, based on the recommended operation reference data acquired by the reference data acquisition section 108.

[Display Device]

The display device 30 is a monitor, such as a liquid crystal display, and is also referred to as an output device. The display device 30 displays the recommended operation information output by the output information processor 106. In addition, the display device 30 may display an endoscope image, an endoscope insertion shape, attention state information, etc.

The operation of the endoscope system 1, for example, an operation in a colonoscopy, will be described.

The insertion section 11 of the endoscope 10 is inserted into the intestinal tract of the colon from the anus to the rectum, colon, and cecum by an operator. The insertion section 11 advances through the intestinal tract while bending following the shape of the intestine. An optical image of the object obtained by an observation optical system of the distal hard section 12 is converted to an electric signal by the imaging element 15. The image processor 102 generates an image signal based on an electric signal output from the imaging element 15 and causes the display device 30 to display an endoscope image of the inside of the intestinal tract based the generated image signal.

(Example of Processing by Recommended Operation Presentation System)

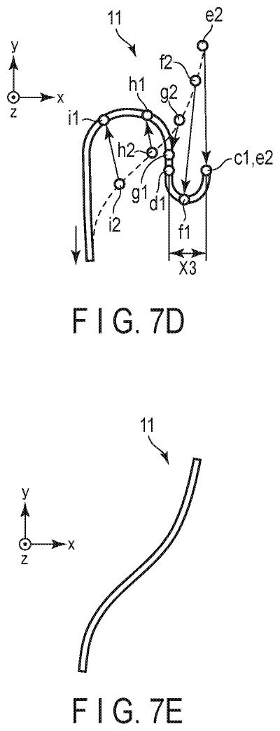

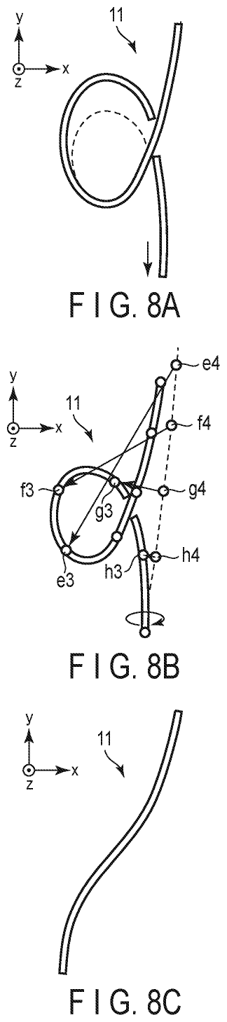

An example of the processing by the control device 100 of the endoscope system 1 as the recommended operation presentation system will be described with reference to FIGS. 4A and 4B.

In step S11, the shape arithmetic operator 103 calculates a shape of the insertion section 11 based on the intensity information of a magnetic field received from the receiving antenna 23. The result of the operation is conveyed as shape information to the attention state/operation control information arithmetic operator 104.

In step S12, the attention state/operation control information arithmetic operator 104 detects attention state information, such as a characteristic shape (e.g., an N-loop or an α-loop), an insertion length, etc., based on the shape information.

In step S13, the recommended operation arithmetic operator 105 acquires recommended operation reference data as shown in FIG. 5, which is preliminarily stored in the storage 107, through the reference data acquisition section 108, and refers to the recommended operation reference data to determine whether or not the attention state information detected in step S12 applies to the reference attention state information of the recommended operation reference data with reference to the recommended operation reference data. With this configuration, the recommended operation arithmetic operator 105 determines whether or not the recommended operation information is necessary.

Alternatively, as shown in FIG. 6, reference attention state information (such as a substantially linear shape, etc.) in the case where recommended operation information is unnecessary is included in recommended operation reference data, and recommended operation necessity determination information, which is preliminarily set so as to correspond to the reference attention state information, is preliminarily stored in the storage 107, and the recommended operation arithmetic operator 105 may refer to the attention state information and the reference attention state information to determine whether or not the recommended operation information is necessary based on the corresponding recommended operation necessity determination information. For example, determining whether or not recommended operation information is necessary based on the recommended operation necessity determination information and, only when determining that the recommended operation information is necessary, calculating operation control information to output recommended operation information reduces displaying unnecessary information for the operator. In other words, it becomes easy to obtain necessary information from the display device 30.

If it is determined that the attention state information does not apply to the reference attention state information, or if it is determined from the recommended operation necessity determination information that the recommended operation information is unnecessary even if attention state information applies to the reference attention state information (step S13—No), then the processing proceeds to step S20. If it is determined that the attention state information applies to the reference attention state information, or if it is determined from the recommended operation necessity determination information that the recommended operation information is necessary even if the attention state information does not apply to the reference attention state information (step S13—Yes), then the processing proceeds to step S14.

In step S14, the attention state/operation control information arithmetic operator 104 reads target shape information corresponding to the reference attention state information determined to be applied in at least step S13 from the recommended operation reference data, and calculates the operation control information based on at least the target shape information and the shape information calculated in step S11. The operation control information is conveyed to the recommended operation arithmetic operator 105. The operation control information is stored in the storage 107.

In step S15, the recommended operation arithmetic operator 105 calculates recommended operation information including the operation direction read from the recommended operation reference data based on the reference attention state information corresponding to the attention state information calculated in step S12 and the operation amount based on the operation control information calculated in step S14. The recommended operation information is conveyed to the output information processor 106.

In step S16, the output information processor 106 processes the recommended operation information as the output information and causes the display device 30 to display the recommended operation information. The operator performs a bending operation, a twisting operation, an insertion/extraction operation, or the like in the predetermined operation amount of the insertion section 11 in accordance with the displayed recommended operation information.

In step S17, the shape arithmetic operator 103 calculates the current shape of the insertion section 11 based on the intensity information of the magnetic fields received from the receiving antenna 23. The result of the calculation is conveyed as the current shape information to the recommended operation arithmetic operator 105.

In step S18, the recommended operation arithmetic operator 105 determines whether or not the current shape of the insert section 11 fits the target shape relating to the target shape information referred to in step S14, based on the current shape information acquired in step S17. That is, the recommended operation arithmetic operator 105 determines whether or not an operation in accordance with the recommended operation information for the current attention state (i.e., an attention state relating to the attention state information detected in step S12) has been completed. If the recommended operation is determined to have been completed (step S18—YES), the processing proceeds to step S19. Otherwise (step S18—NO), the processing proceeds to step S21.

In step S19, the recommended operation arithmetic operator 105 reads the recommended operation continuation information corresponding to the reference attention state information determined to be applied in step S13, and determines whether the recommended operation continuation information for the current attention state is “completed” or “continue”. If the processing is “completed” (step S19—YES), the processing proceeds to step S20. If the recommended operation continuation information is “continue” (step S19—NO), the process returns to step S14.

In step S20, the recommended operation arithmetic operator 105 determines whether or not the insertion operation has been completed, i.e., whether or not the insertion section 11 has reached the cecum. The determination may be made based on the shape of the insertion section 11 or the history of the shape (e.g., time-varying change in shape of the insertion section 11), the position of the distal end, or the like.

Alternatively, it may be configured that the endoscope system 1 is provided with an input device, such as a switch or a keyboard, and the insertion operation is determined to have been completed when the switch is pressed or a predetermined entry is made through the keyboard, by the operator's determination. Also, the insertion operation may be determined to have been completed when the cecum is detected in an endoscope image by means of an image recognition technique. If the insertion operation has been completed (step S20—YES), then the processing ends. If the insertion operation has not been completed (step S20—NO), then the processing returns to step S11.

If it is determined that the operation in accordance with the recommended operation information for the current attention state has not been completed in step S18 (step S18—NO), then the processing proceeds to step S21. In step S21, the attention state/operation control information arithmetic operator 104 acquires the current shape information in step S17 and detects attention state information based on the current shape information, in the same manner as in step S12.

In step S22, the attention state/operation control information arithmetic operator 104 calculates operation control information from at least the target shape information and the current shape information calculated in step S17 in the same manner as in step S14. The operation control information will be stored in the storage 107.

In step S23, the recommended operation arithmetic operator 105 determines whether or not the operation performed by the operator conforms to the recommended operation. Specifically, the recommended operation arithmetic operator 105 determines whether or not the direction of the operation performed by the operator (operation direction by the operator) and the operation direction of the recommended operation information (recommended operation direction) conform to each other, based on, e.g., comparison between, a value of the operation control information calculated in step S22 immediately before this step and a value of the operation control information calculated in step S14 (or step S22) that has been performed in the step one step prior to this step relating to the calculation of the operation control information. When the operation direction by the operator is determined to conform to the operation direction of the recommended operation information (step S23—YES), the processing proceeds to step S24. When the operation direction by the operator is determined not to conform to the operation direction of the recommended operation information (step S23—NO), the processing proceeds to step S25. When the reference attention state information corresponding to the attention state information detected in step S21 differs from the reference attention state information referred to in the previous step S12 relating to the reference attention state information, even though the insertion section has not reached the target shape, the operation performed by the operator is determined not to conform to the recommended operation.

In step S24, the recommended operation arithmetic operator 105 updates the operation amount based on the operation control information calculated in step S22. Then, the processing proceeds to step S16, and the output information processor 106 processes recommended operation information including an operation direction and an updated operation amount as output information and causes the display device 30 to display the recommended operation information. After step S16, the above-mentioned processing of step S17 and subsequent steps is performed.

On the other hand, in step S25, the recommended operation arithmetic operator 105 determines whether or not the operation in accordance with the recommended operation information can be performed. When it is determined that the operation in accordance with the recommended operation information cannot be performed (step S25—NO), the processing proceeds to step S14. In step S14, the attention state/operation control information arithmetic operator 104 calculates operation control information different than the operation control information including the operation that has not been performed. After step S14, the above-mentioned processing of step S15 and subsequent steps is performed.

On the other hand, when it is determined that the operation in accordance with the recommended operation information can be performed (step S25—YES), the processing proceeds to step S26. In step S26, the recommended operation arithmetic operator 105 corrects the recommended operation information. For example, if the operation direction by the operator is opposite to the recommended operation direction, then the recommended operation arithmetic operator 105 corrects the operation amount based on the operation control information calculated in step S22 and determines to output an alert indicating that the operation direction is opposite to the recommended operation direction. When the attention state information is changed and corresponding reference attention state information is changed, the recommended operation arithmetic operator 105 refers to the recommended operation reference data again and calculates an operation direction and an operation amount from the applied reference attention state information again. The recommended operation arithmetic operator 105 may determine to output, to the display device 30, an alert indicating that the operation performed by the operator does not conform to the recommended operation. After step S26, the processing proceeds to step S16.

In step S16 after step S26, the output information processor 106 processes the recommended operation information and the alert as output information and causes the display device 30 to display the recommended operation information. After that, the above-mentioned processing of step S17 and subsequent steps is performed.

(Example of Processing During Releasing N-Loop)

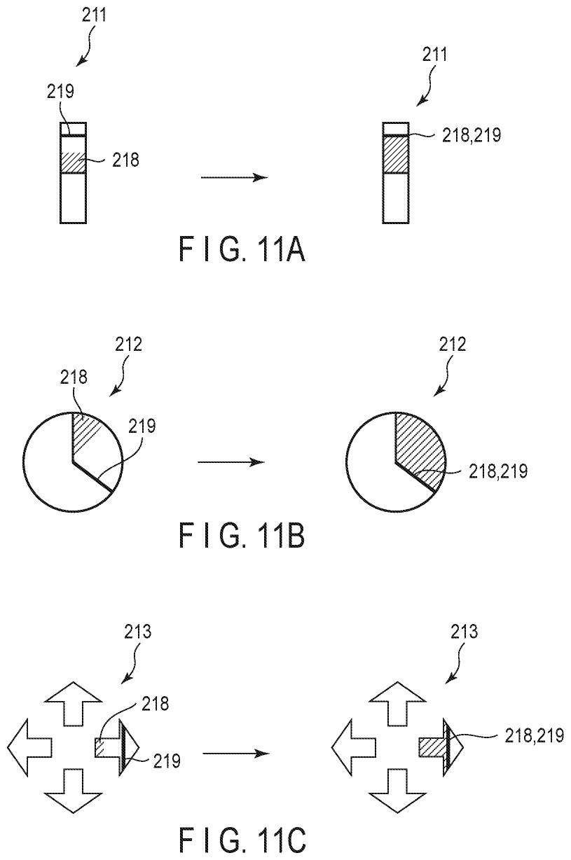

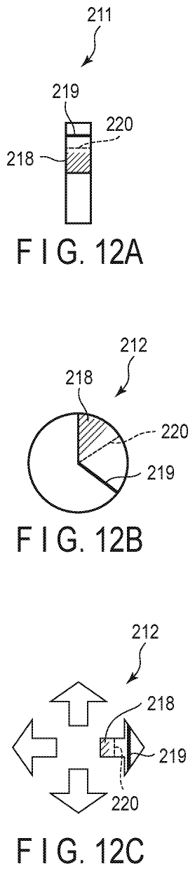

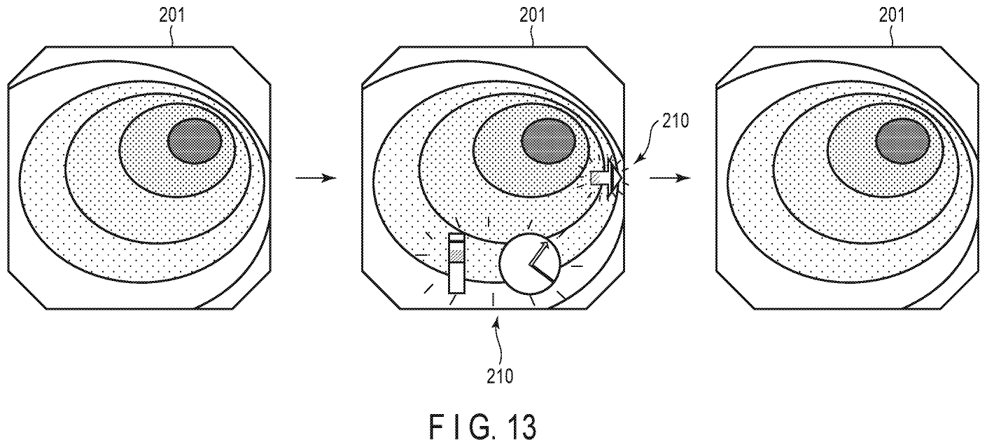

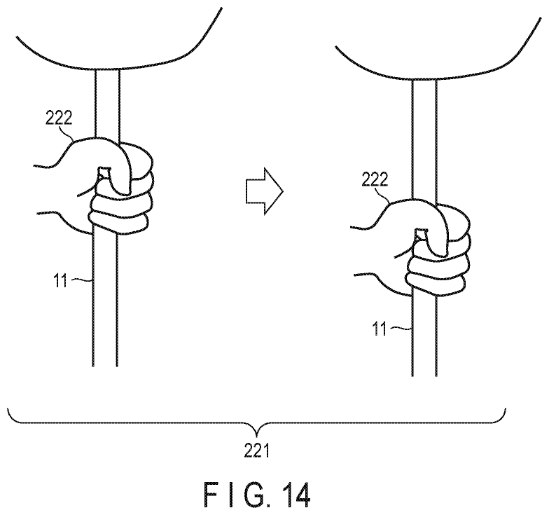

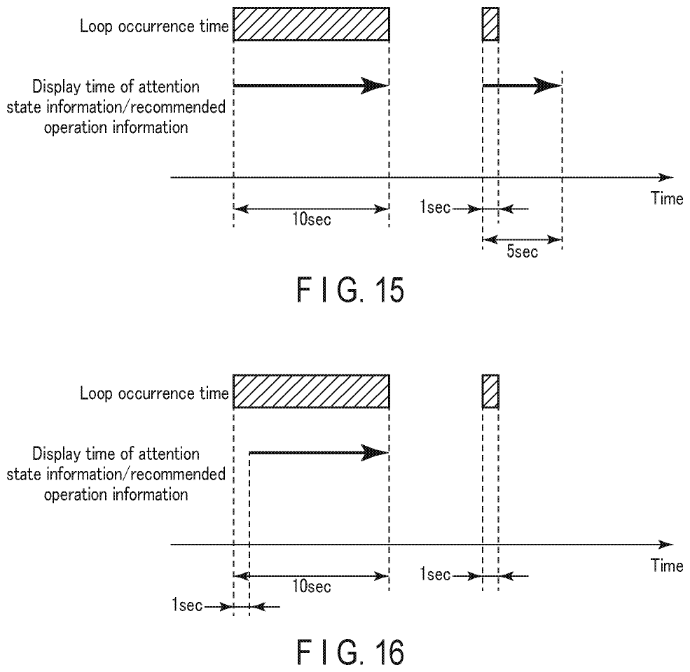

The series of processing shown in FIGS. 4A and 4B have been described. Hereinafter, the processing in the recommended operation presentation system relating to releasing of an N-loop during insertion of the insertion section 11 will be specifically described with reference to flowcharts of FIGS. 4A and 4B and the recommended operation reference data of FIG. 5 along with FIGS. 7A through 7E.

In step S11, the shape arithmetic operator 103 calculates first shape information of the insertion section 11. In step S12, the attention state/operation control information arithmetic operator 104 detects an N-loop having the generally ρ-character as shown in FIG. 7A as first attention state information, based on the first shape information. In other words, the first attention state is “a generally ρ-character shape with an a1-b1 distance of X1 or more (X1≥10 cm).” Here, a1 and b1 are points in the x-y plane shown in FIG. 7A. a1 is a point of an N-loop formed by the insertion section 11 where a y-coordinate is the largest (y-coordinate: ya1), and b1 is a point of the N-loop formed by the insertion section 11 where a y-coordinate is the smallest (y-coordinate: yb1).

In step S13, the recommended operation arithmetic operator 105 refers to the reference attention state information shown in FIG. 5 to determine whether or not the first attention state information detected in step S12 applies to the reference attention state information of the recommended operation reference data. In this case, since the first attention state information applies to the item (i) of the reference attention state information in the recommended operation reference data, “the generally ρ-character shape with the a1-b1 distance of 10 cm or more” (step S13—YES), the processing proceeds to step S14. In other words, the recommended operation information is determined to be necessary in step S13, and the processing proceeds to step S14.

In step S14, the attention state/operation control information arithmetic operator 104 reads target shape information corresponding to the item (i) of the recommended operation reference data. Here, “a generally ρ-character shape with an a2-b2 distance of less than 10 cm” is read out. Here, a2 and b2 are points in the x-y plane shown in FIG. 7A. a2 is a point where a y-coordinate of an N-loop having a target shape is the largest (y-coordinate: ya2), and b2 is a point where the N-loop having the target shape is the smallest (y-coordinate: yb2). The attention state/operation control information arithmetic operator 104 calculates a difference in size between the generally ρ-character shape according to the first operation control information and the generally ρ-character shape according to the target shape information to set the difference to first operation control information. In FIG. 7A, the generally ρ-character shape according to the first shape information is indicated by an outlined solid line, and the generally ρ-character shape according to the target shape information is indicated by a dotted line (the same applies to the following figures). The attention state/operation control information arithmetic operator 104 sets, for example, as shown in FIG. 7A, a difference Δya1b1-Δya2b2 between a difference Δya1b1 between a y-coordinate ya1 at the point a1 and a y-coordinate yb1 at the point b1 and a difference Δya2b2 between a y-coordinate ya2 at the point a2 and a y-coordinate yb2 at the point b2 to the first operation control information.

In step S15, the recommended operation arithmetic operator 105 reads a pulling operation as an operation direction corresponding to the item (i) of the recommended operation reference data. The recommended operation arithmetic operator 105 calculates the operation amount of the pulling operation based on the first operation control information Δya1b1-Δya2b2. Here, the operation amount calculated based on the first operation control information is a direct operation amount, such as how many centimeters the loop should be pulled. In this way, the recommended operation arithmetic operator 105 calculates, as recommended operation information, first recommended operation information including a first recommended operation direction t that is a pulling operation based on the first attention state information and a first recommended operation amount based on the first operation control information. FIG. 7A shows a downward arrow indicating an operation of pulling the loop toward the hand side of the insertion section 11.

In step S16, the output information processor 106 processes the first recommended operation information as output information and causes the display device 30 to display the output information. The operator performs a pulling operation in a predetermined operation amount of the insertion section 11 in accordance with the displayed recommended operation information.

In step S17, the shape arithmetic operator 103 calculates the current shape of the insertion section 11. In step S18, the recommended operation arithmetic operator 105 determines whether or not the operation in accordance with the recommended operation information has been completed until the current shape of the insertion section 11 fits a target shape according to the target shape information referred to in step S14, based on the shape information relating to the current shape. For example, when a value of the first operation control information calculated as described above based on the shape information in this step is 0 or less than 0, the recommended operation arithmetic operator 105 determines that the recommended operation has been completed. If the operator performs a pulling operation (i.e., an insertion operation in accordance with the recommended operation direction) by little by little, then processing from step S21 to step S24 is repeated until it is determined that the operation has been completed (step S18—YES), and the processing proceeds to step S19.

In step S19, the recommended operation arithmetic operator 105 determines whether the recommended operation continuation information corresponding to the item (i) of the recommended operation reference data is “completed” or “continue”. Here, since the processing is “continue” (step S19—NO), the processing returns to step S14. When the recommended operation continuation information is “continue”, the processing proceeds to step S14 and continues calculations of recommended operation information based on new attention state information, because an item (ii) following the item (i) is prepared in the recommended operation reference data.

Here, the shape information relating to the current shape calculated in step S17 described above is set to second shape information. The second shape information corresponds to the target shape information of the item (i) of the recommended operation reference data, “a generally ρ-character shape with an a2-b2a distance of less than 10 cm”.

In step S14, the attention state/operation control information arithmetic operator 104 reads the reference attention state information, operation direction, and target shape information, each corresponding to the item (ii) next to the item (i) of the recommended operation reference data. In other words, in this case, “a generally ρ-character/N-character shape with an a1-b1 distance of less than 10 cm,” “clockwise twisting,” and “an N-character shape with θ being within ±10°” are read out. Here, the read reference attention state information is second attention state information. The attention state/operation control information arithmetic operator 104 calculates a difference in angle of coordinates between the generally ρ-character shape according to the second attention state information and the N-character shape according to the target shape information to set the difference to second operation control information.

Here, the second operation control information will be described in detail. As shown in FIG. 7B, an x-y plane is set, and points c1, c2, d1, and d2 are set. The points c2 and d2 are respectively points of a target shape relating to the points c1 and d1 of the generally ρ-character shape according to the second shape information in the x-y plane shown in FIG. 7B. The c1 is a point (y-coordinate: yc1) of the distal end of the insertion section 11 having the generally ρ-character shape according to the second shape information. The d1 is a point (y-coordinate: yd1) where the y-coordinate in the N-loop having the generally ρ-character shape formed by the insertion section 11 is equal to the y-coordinate yc1 at the point c1. The c2 is a point (y-coordinate: yc2) of the distal end of the insertion section 11 having the target shape. The d2 is the same point (y-coordinate: yd2) as the point of d1. It is considered that the point c1 performs a generally circular movement in an x-z plane as shown in FIG. 7C by a clockwise twisting operation. Assuming that the point c1 performs a circular movement around the point d1 by the twisting operation to move to the point c2 on the x-axis, an angle θ formed between a line connecting the points c2 and d2, i.e., an x-axis, and a line connecting the points d1 and c1 is determined is determined to be set to second operation control information.

In step S15, the recommended operation arithmetic operator 105 reads the clockwise twisting operation as an operation direction corresponding to the item (ii) of the recommended operation reference data. Also, the recommended operation arithmetic operator 105 calculates an operation amount of the twisting operation based on the second operation control information. Here, the angle θ, which is the second operation control information, may be directly set to a rotation operation amount. As described above, the recommended operation arithmetic operator 105 calculates, as recommended operation information, the second recommended operation information including a second recommended operation direction that is a clockwise twisting operation based on the second attention state information and a second recommended operation amount based on the second operation control information. FIG. 7B shows an arrow indicating the clockwise twisting operation on the hand side of the insertion section 11.

In step S16, the output information processor 106 processes the second recommended operation information as output information and causes the display device 30 to display the second recommended operation information. The operator performs the clockwise twisting operation of the predetermined operation amount of the insertion section 11 in accordance with the displayed recommended operation information.

In step S17, the shape arithmetic operator 103 calculates the current shape of the insertion section 11. In step S18, the recommended operation arithmetic operator 105 determines whether or not the operation in accordance with the recommended operation information has been completed until the current shape of the insertion section 11 fits a target shape according to the target shape information referred to in step S14, based on the shape information relating to the current shape. For example, when the value of the second operation control information calculated as described above based on the shape information at this step becomes 0 or within a predetermined range including 0, it is determined that the recommended operation has been completed. If the operator performs a clockwise twisting operation (i.e., an operation in accordance with a recommended operation direction) by little by little, then processing from step S21 to step S24 is repeated until it is determined that the operation has been completed (step S18—YES). If the operation is determined to have been completed, then the processing proceeds to step S19.

In step S19, the recommended operation arithmetic operator 105 determines whether recommended operation continuation information corresponding to the item (ii) of the recommended operation reference data is “completed” or “continue”. Here, since the processing is “continue” (step S19—NO), the processing returns to step S14. When the recommended operation continuation information indicates “continue,” an item (iii) following the item (ii) is prepared in the recommended operation reference data. Therefore, the processing proceeds to step S14 and continues calculations of recommended operation information based on new attention state information.

Here, the shape information of the current shape calculated in step S17 described above is set to third shape information. The third shape information corresponds to target shape information of the item (iii) of the recommended operation reference data, “an N-character shape with θ being within ±10°”.

In step S14, the attention state/operation control information arithmetic operator 104 reads reference attention state information and target shape information corresponding to the item (iii) next to the item (ii) of the recommended operation reference data. In other words, in this case, “an N-character shape with θ being within ±10°” and “coordinate data of a generally straight line” is read. Here, the read reference attention state information is the third attention state information. Then, the attention state/operation control information arithmetic operator 104 calculates a difference between the N-character shape according to the third attention state information and the generally straight line shape according to the target shape information to set the difference to third operation control information.

FIG. 7D shows, in an x-y plane, the insertion section 11 with a distance between c1 and d1 being X3, which has an N-character shape according the third shape information. FIG. 7D shows discretional points e1, f1, g1, h1, and it of an N-character shape according to the third shape information of the insertion section 11, and discretional points e2, f2, g2, h2, and i2 of a target shape relating to the above-mentioned points, respectively. The third operation control information is, for example, a total value of differences in x-y coordinate data of the respective points. The arrows respectively indicated between point pairs e1-e2, f1-f2, g1-g2, h1-h2, and it-i2 shown in FIG. 7D conceptually represent the third operation control information.

In step S15, the recommended operation arithmetic operator 105 reads a pulling operation as an operation direction corresponding to the item (iii) of the recommended operation reference data. The recommended operation arithmetic operator 105 calculates the operation amount of the pulling operation based on the third operation control information. For example, when the total value of differences of the x-y coordinate data of the respective points is the third operation control information, the operation amount calculated based on the third operation control information is an indirect operation amount represented by a total value of the differences rather than a direct operation amount for the pulling operation, such as how many centimeters the loop should be pulled. As described above, the recommended operation arithmetic operator 105 calculates, as recommended operation information, third recommended operation information including a third recommended operation direction that is a pulling operation based on the third attention state information and a third recommended operation amount based on the third operation control information. FIG. 7D shows a downward arrow indicating an operation of pulling the loop toward the hand side of the insertion section 11.

In step S16, the output information processor 106 processes the third recommended operation information as output information and causes the display device 30 to display the third recommended operation information. The operator performs the pulling operation in a predetermined operation amount of the insertion section 11 in accordance with the displayed recommended operation information.

In step S17, the shape arithmetic operator 103 calculates the current shape of the insertion section 11. In step S18, the recommended operation arithmetic operator 105 determines whether or not the operation in accordance with the recommended operation information has been completed until the current shape of the insertion section 11 fits a target shape according to the target shape information referred to in step S14, based on the shape information relating to the current shape. For example, as a value of the third operation control information approaches 0, the insertion section 11 has a shape closer to the target shape. However, since usually it is not necessary for the operation control information to exactly coincide with the target shape, a threshold for the value of the third operation control information is set, and if the value of the third operation control information takes a value less than the threshold, it is determined that the operation for the target shape has been completed, that is, the recommended operation for the N-loop has been completed, so that the N-loop is nearly straightened, as shown in FIG. 7E. Here, it is determined that the operation has not been completed (step S18—NO), and the processing proceeds to step S21.

In step S21, the attention state/operation control information arithmetic operator 104 acquires the current shape information in step S17 and detects attention state information based on the current shape information in the same manner as in step S12.

In step S22, the attention state/operation control information arithmetic operator 104 calculates operation control information from a difference between the current shape information in step S17 and the target shape information in the same manner as in step S14.

In step S23, the recommended operation arithmetic operator 105 determines whether or not the operation performed by the operator conforms to the recommended operation. Here, the recommended operation arithmetic operator 105 calculates an increase or decrease of a value of the operation control information based on the value of the operation control information calculated in step S22 immediately before this step and a value of the operation control information calculated in step S14 that has been performed one step prior to this step relating to the calculation of the operation control information, and determines whether or not the operation direction by the operator and the recommended operation direction conform to each other. For example, if the operation direction by the operator and the recommended operation direction conform to each other, then the value of the operation control information has decreased. On the other hand, if these directions do not match, the value of the operation control information has increased. Here, it is determined that the value of the operation control information has increased and these directions do not match (step S23—NO), and the processing proceeds to step S25.

In step S25, the recommended operation arithmetic operator 105 determines whether or not the recommended operation in accordance with the recommended operation information cannot be performed. This is determined based, for example, on a fact that the shape information obtained from the shape arithmetic operator 103 has not been changed for a predetermined time, or recognition through an endoscope image by an image recognition technology that an endoscope image has not been changed for a predetermined time, or the insertion is difficult. Hereinafter, examples of two ways of processing will be described, i.e., a case of a state where the recommended operation in accordance with the recommended operation information can be performed (step S25—YES) and a case of a state where the recommended operation cannot be performed (step S25—NO).

(Example of the Case of the State where the Recommended Operation in Accordance with the Recommended Operation Information can be Performed)

In the case (step S25—YES) of the state where the recommended operation in accordance with the recommended operation information can be performed, the processing proceeds to step S26.

In step S26, the recommended operation arithmetic operator 105 calculates the recommended operation information again to correct the recommended operation information. For example, if the operation direction by the operator is opposite to a recommended operation direction, the recommended operation arithmetic operator 105 increases the operation amount based on the operation control information calculated in step S22. Also, the recommended operation arithmetic operator 105 determines to output an alert that the operation direction is opposite to the display device 30. Then, the processing proceeds to step S16.

In step S16, the output information processor 106 processes the recommended operation information including the increased operation amount and the alert as output information and causes the display device 30 to display the output information.

In step S17, the shape arithmetic operator 103 calculates the current shape of the insertion section 11. In step S18, the recommended operation arithmetic operator 105 determines whether or not the operation in accordance with the recommended operation information has been completed until the current shape of the insertion section 11 fits a target shape according to the target shape information referred to in step S14, based on the shape information relating to the current shape. Here, it is determined that the operation has not been completed (step S18—NO), and the processing proceeds to step S21.

After going through steps S21 and S22, in step S23, the recommended operation arithmetic operator 105 determines whether or not the operation performed by the operator conforms to the recommended operation. Here, it is determined that the operation performed by the operator conforms to the recommended operation (step S23—YES), and the processing proceeds to step S24.

In step S24, the recommended operation arithmetic operator 105 calculates the operation amount based on the operation control information calculated in step S22 again to update the operation amount. Here, the operation amount is reduced. Then, the processing proceeds to step S16, and the output information processor 106 processes the recommended operation information including the operation direction and the updated operation amount as output information and causes the display device 30 to display the output information. After going through step S17 as described above, in step S18, the recommended operation arithmetic operator 105 determines whether or not an operation in accordance with the recommended operation information has been completed until the current shape of the insertion section 11 fits a target shape according to the target shape information referred to in step S14, based on the shape information relating to the current shape of the insertion section 11. Here, it is determined that the operation has been completed (step S18—YES), and the processing proceeds to step S19.

In step S19, the recommended operation arithmetic operator 105 determines whether the recommended operation continuation information corresponding to the item (iii) of the recommended operation reference data is “completed” or “continue”. Here, since the processing is “completed” (step S19—YES), the processing proceeds to step S20. That is, when the recommended operation continuation information is “completed”, the processing proceeds to step S20 because no data following the item (iii) is present in the recommended operation reference data.

In step S20, the attention state/operation control information arithmetic operator 104 determines whether or not the insertion operation has been completed. If the insertion operation is determined to not have been completed (step S20—NO), then the processing returns to step S11, and the processing of step S11 and subsequent processing is repeated. If the insertion operation is determined to have been completed (step S20—YES), then the processing ends.

(Example of the Case of the State where the Recommended Operation in Accordance with the Recommended Operation Information Cannot be Performed)

In the case of the state where the recommended operation in accordance with the recommended operation information cannot be performed (step S25—NO), the processing proceeds to step S14. This is, for example, a case where an insertion operation as the recommended operation information cannot be performed, resulting from the condition of an object such as adhesion or the like.

In step S14, the attention state/operation control information arithmetic operator 104 calculates operation control information different from the recommended operation information including the operation that has not been performed. For example, in the case where an N-loop cannot be straightened resulting from synechia of the large intestine, the attention state/operation control information arithmetic operator 104 calculates operation control information based on the recommended operation reference data including “pushing” as an operation direction. This is because a pushing operation is effective when the straightening of an N-loop cannot be performed by a pulling operation due to adhesion or the like, although straightening of an N-loop by pulling it causes less burden on an object, e.g., a patient.

As shown in FIG. 5, the storage 107 stores an item (iv) of the recommended operation reference data for use in the case where an operation in accordance with recommended operation information referred to in the item (iii) of the recommended operation reference data has not been performed. In the item (iv), for example, the reference attention state information, target shape information, and recommended operation continuation information is the same as the information in the item (iii), but the operation direction is “pushing”, which is different than “puling” in the item (iii). The attention state/operation control information arithmetic operator 104 refers to the item (iii) in priority to the item (iv) of the recommended operation reference data; however, when an operation in accordance with the recommend operation information referred to in the item (iii) of the recommended operation reference data has not been performed, attention state/operation control information arithmetic operator 104 refers to the item (iv).

In step S15, the recommended operation arithmetic operator 105 calculates recommended operation information including, as a recommended operation, a pushing operation based on the attention state information and an operation amount based on the operation control information. After going through steps S16 and S17, in step S18, the recommended operation arithmetic operator 105 determines whether or not an operation in accordance with the recommended operation information has been completed until the current shape of the insertion section 11 fits a target shape according to the target shape information referred to in step S14, based on the shape information relating to the current shape. If the operator performs a pushing operation (i.e., an operation in accordance with a recommended operation direction) by little by little, then processing from step S21 to step S24 is repeated until it is determined that the operation has been completed. When it is determined that the operation has been completed (step S18—YES), the processing proceeds to step S19.

In step S19, the recommended operation arithmetic operator 105 determines whether recommended operation continuation information corresponding to the item (iv) of the recommended operation reference data is “completed” or “continue”. Here, since the processing is “completed” (step S19—YES), the processing proceeds to step S20.

In step S20, the attention state/operation control information arithmetic operator 104 determines whether or not the insertion operation has been completed. If the insertion operation is determined to not have been completed (step S20—NO), then the processing returns to step S11, and the processing of step S11 and subsequent processing is repeated. If the insertion operation is determined to have been completed (step S20—YES), the processing ends.

(Example of Processing During Releasing α-Loop)

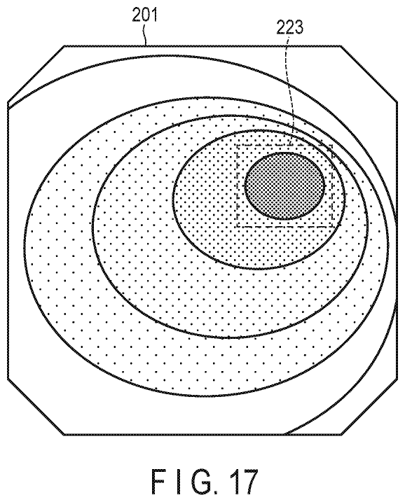

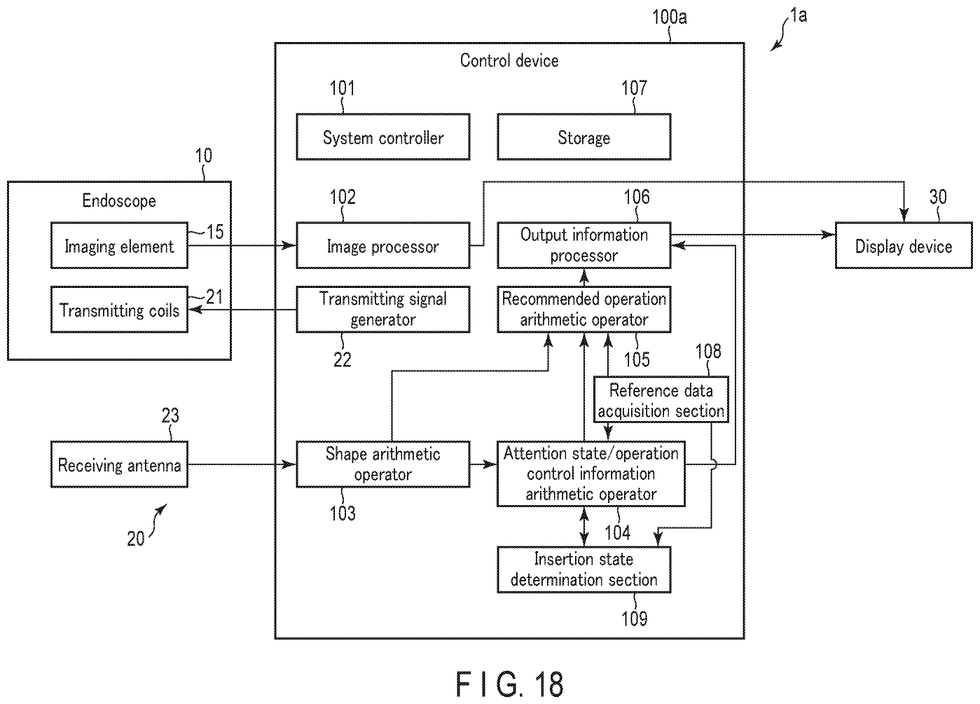

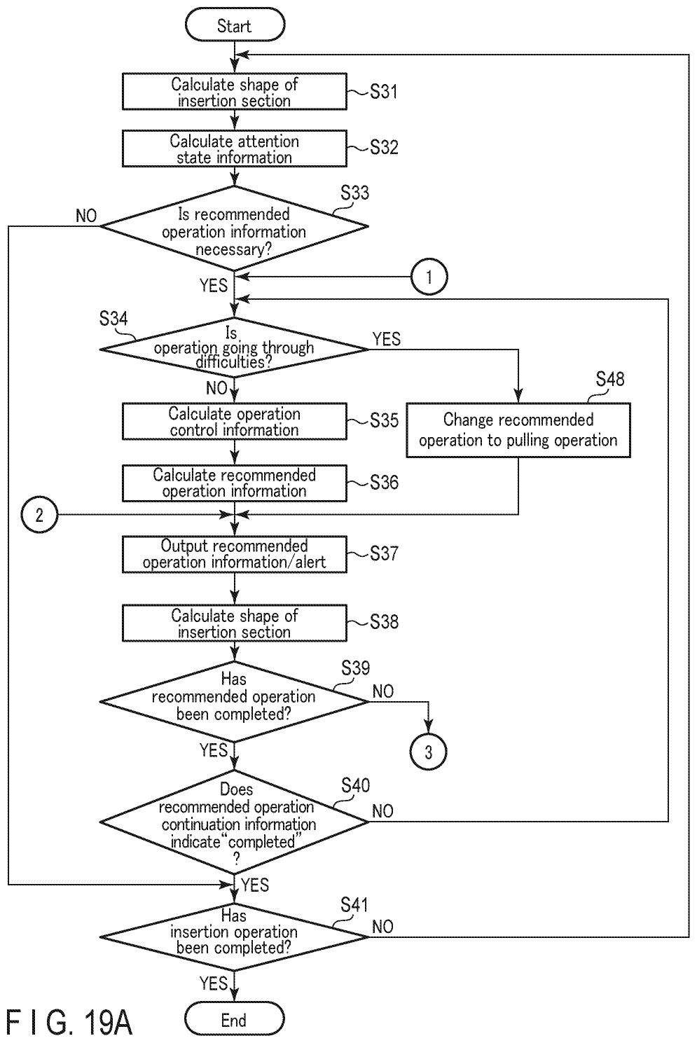

Hereinafter, the processing relating to release of an α-loop during insertion of the insertion section 11 in the recommended operation presentation system will be specifically described with reference to the flowcharts of FIGS. 4A and 4B, and the recommended operation reference data shown in FIG. 5 along with FIGS. 8A and 8C.

In step S11, the shape arithmetic operator 103 calculates fourth shape information of the insertion section 11. In step S12, the attention state/operation control information arithmetic operator 104 detects an α-loop as shown in FIG. 8A as fourth attention state information, based on the fourth shape information. In other words, a fourth attention state is “an α-loop having a loop area of X4 or more (X4≥300 cm²). Here, the loop area is an area of a loop shape that is made when an approximate plane for coordinates in a range forming a loop is calculated and the loop is projected onto the approximation plane. For example, if the insertion section 11 is spiral in a three-dimensional space, the area of the loop cannot be obtained without being projected, so that such a technique is employed.

In step S13, the recommended operation arithmetic operator 105 refers to the reference attention state information shown in FIG. 5 to determine whether or not the fourth attention state information detected in step S12 applies to the reference attention state information of the recommended operation reference data. Here, since the fourth attention state information applies to the reference attention state information of an item (v) of the recommended operation reference data, i.e., “an α-loop having a loop area of 300 cm² or more” (step S13—YES), the processing proceeds to step S14.

In step S14, the attention state/operation control information arithmetic operator 104 reads target shape information corresponding to the item (v) of the recommended operation reference data. Herein, “an α-loop having a loop area of less than 300 cm²” is read out. In FIG. 8A, a shape according to the fourth shape information is indicated by an outlined solid line, and a target shape of the inversion section 11 according to the fourth shape information is indicated by a dotted line. The attention state/operation control information arithmetic operator 104 sets, to fourth operation control information, a difference between the area X4 of the α-loop according to the fourth attention state information and the area 300 cm² of the α-loop according to the target shape information. The area of the α-loop itself may be set to the fourth operation control information.

In step S15, the recommended operation arithmetic operator 105 reads a pulling operation as an operation direction corresponding to the item (v) of the recommended operation reference data. The recommended operation arithmetic operator 105 calculates the operation amount of the pulling operation based on the fourth operation control information. For example, when the difference in the area is the fourth operation control information, the operation amount calculated based on the fourth operation control information is an indirect operation amount represented by the difference in the area rather than a direct operation amount for the pulling operation, such as how many centimeters the loop should be pulled. As described above, the recommended operation arithmetic operator 105 calculates, as recommended operation information, fourth recommended operation information including a fourth recommended operation direction as a pulling operation based on the fourth attention state information and a fourth recommended operation amount based on the fourth operation control information. FIG. 8A shows a downward arrow indicating an operation of pulling the loop toward the hand side of the insertion section 11.

In step S16, the output information processor 106 processes the fourth recommended operation information as output information and causes the display device 30 to display the output information. The operator performs the pulling operation in a predetermined operation amount of the insertion section 11 in accordance with the displayed recommended operation information.

In step S17, the shape arithmetic operator 103 calculates the current shape of the insertion section 11. In step S18, the recommended operation arithmetic operator 105 determines whether or not the operation in accordance with the recommended operation information has been completed until the current shape of the insertion section 11 fits a target shape according to the target shape information referred to in step S14, based on the shape information relating to the current shape. For example, when a value of the fourth operation control information calculated as described above based on the current shape information is 0 or less than 0, it is determined that the recommended operation has been completed. If the operator performs a pulling operation (i.e., an insertion operation in accordance with a recommended operation direction) by little by little, then processing from step S21 to step S24 is repeated until it is determined that the operation has been completed (step S18—YES), and the processing proceeds to step S19.

In step S19, the recommended operation arithmetic operator 105 determines whether the recommended operation continuation information corresponding to the item (v) of the recommended operation reference data is “completed” or “continue”. Here, since the recommended operation continuation information indicates “continue” (step S19—NO), the processing returns to step S14. Here, the shape information of the current shape calculated in step S17 described above is set to fifth shape information. The fifth shape information corresponds to the target shape information of the item (vi) of the recommended operation reference data, “an α-loop having a loop area of less than 300 cm²”.

In step S14, the attention state/operation control information arithmetic operator 104 reads reference attention state information and target shape information corresponding to the item (vi) of the recommended operation reference data. Namely, here, “an α-loop having a loop area of less than 300 cm²” and “coordinate data of a generally straight line” are read out. Here, the read reference attention state information is fifth attention state information. The attention state/operation control information arithmetic operator 104 calculates a difference between the shape of the α-loop relating to the fifth attention state information and the generally straight line according to the target shape information to set the difference to fifth operation control information.

FIG. 8B shows, in the x-y plane, the insertion section 11 in which an α-loop having a loop area of X5 is formed, which is a shape according the fifth shape information. FIG. 8B shows discretional points of a shape according to the fifth shape information, e3, f3, g3, and h3, and points of a target shape relating to each of the above-mentioned points, e4, f4, g4, and h4. The fifth operation control information is, for example, a total of differences of x-y-coordinate data of the respective points. The arrows respectively indicated between point pairs e3-e4, f3-f4, g3-g4, and h3-h4, shown in FIG. 8B conceptually represent the fifth operation control information.

In step S15, the recommended operation arithmetic operator 105 reads the clockwise twisting operation as an operation direction corresponding to the item (vi) of the recommended operation reference data. Also, the recommended operation arithmetic operator 105 calculates the operation amount of the clockwise twisting operation based on the fifth operation control information. For example, when a total value of the x-y-coordinate data of the respective points is the fifth operation control information, the operation amount calculated based on the fifth operation control information is an indirect operation amount represented by the total value of the differences rather than a direct operation amount for the clockwise twisting operation, such as how many degrees the loop should be twisted. In this way, the recommended operation arithmetic operator 105 calculates, as the recommended operation information, fifth recommended operation information including a fifth recommended operation direction that is a clockwise twisting operation based on the fifth attention state information and a fifth recommended operation amount based on the fifth operation control information. FIG. 8B shows an arrow indicating an operation of twisting a loop in a clockwise direction on the hand side of the insertion section 11.

In step S16, the output information processor 106 processes the fifth recommended operation information as output information and causes the display device 30 to display the output information. The operator performs the clockwise twisting operation of the predetermined operation amount of the insertion section 11 in accordance with the displayed recommended operation information.

In step S17, the shape arithmetic operator 103 calculates the current shape of the insertion section 11. In step S18, the recommended operation arithmetic operator 105 determines whether or not an operation in accordance with the recommended operation information has been completed until the current shape of the insertion section 11 fits a target shape according to the target shape information referred to in step S14 (e.g., until the value of the current shape is lower than a threshold relating to the value of the second operation control information). Herein, the operation is determined to have been completed (step S18—YES), and the processing proceeds to step S19.

In step S19, the recommended operation arithmetic operator 105 determines whether the recommended operation continuation information corresponding to the item (vi) of the recommended operation reference data is “completed” or “continue”. Here, since the recommended operation continuation information is “completed” (step S19—YES), the processing proceeds to step S20.

In step S20, the attention state/operation control information arithmetic operator 104 determines whether or not the insertion operation has been completed. If the insertion operation is determined to not have been completed (step S20—NO), then the processing returns to step S11, and the processing of step S11 and subsequent processing is repeated. If the insertion operation is determined to have been completed (step S20—YES), the processing ends.

Herein, for the purpose of straightening an α-loop in which the insertion section distal end side is present on the upper side (refer to FIGS. 3A, 8A, and 8B), a clockwise twisting operation in a predetermined operation amount is performed after an operation of pulling the loop in a predetermined amount. However, for example, for the purpose of releasing an α-loop in which the insertion section distal end side is present on the lower side (refer to FIG. 3B), it is effective to perform a counter-clockwise twisting operation in a predetermined amount after performing an operation of pulling the loop in a predetermined amount. For the purpose of releasing a left-right reversed α-loop (refer to FIG. 3C) in which the insertion section distal end is present on the upper side, it is effective to perform a counter-clockwise twisting operation in a predetermined amount after performing an operation of pulling the loop in a predetermined amount. Furthermore, for the purpose of releasing a left-right reversed α-loop (refer to FIG. 3D) in which the insertion section distal end side is present on the upper side, it is effective to perform a clockwise twisting operation in a predetermined amount after performing an operation of pulling the loop in a predetermined amount. The recommended operation presentation system according to the present embodiment can provide recommended operation information including a most suitable operation direction for straightening of each loop as described above based on the processing flow as described above.

According to the present embodiment, the recommended operation presentation system refers to recommended operation reference data in which reference attention state information, an operation direction, target state information and the like is preliminarily set, so as able to calculate attention state information and operation control information, and further calculate recommended operation information including an operation direction based on the attention state information and an operation amount based on the operation control information. The recommended operation presentation system presents recommended operation information to the operator by calculating and outputting the recommended operation information. This allows the operator to perform an insertion operation based on exact insertion support information. In particular, the present embodiment presents a direct or an indirect operation amount as well as an operation direction, which assisting an operator who is unfamiliar with an insertion operation.

In particular, in the case of a colonoscope, the insertion section 11 is inserted into a movable intestinal tract having bent portions, such as the sigmoid colon and the transverse colon, and thus a situation in which it is difficult to continue the operation as it is, such as in an N-loop and an α-loop, is likely to occur. Even in such a situation, the recommended operation presentation system of the present embodiment allows straightening of an N-loop and an α-loop in accordance with the shape information of the insertion section 11 and recommended operation information including a recommended operation direction and a recommended operation amount that are calculated based on an operation direction and target state information preliminarily set according to each type situation, and in particular, even if the operator is an inexperienced operator, the recommended operation system allows the operator to perform an insertion operation as if the operator is a skilled operator. In addition, when an insertion operation in accordance with recommended operation information is performed, this will present an unnecessary extension of the intestinal tract. Therefore, the recommended operation system can reduce the patient's pain associated with intestinal extension.

In the present embodiment, after outputting recommended information, the recommended operation presentation system determines whether an operation direction by the operator and a recommended operation direction conform to each other and updates or corrects the operation amount as necessary. Such feedback allows the operator to proceed with an operation while recognizing whether the operation as the recommended operation information has been performed and how much of the operation remains.

Also, after outputting recommended information, when the operation direction by the operator is different than the recommended operation direction and it is not a situation where the operation in the recommended operation direction is difficult, the recommended operation presentation system can alert the operator a fact that the operation direction by the operator is different than the recommended operation direction. For example, when the operator misrecognizes the recommended operation direction, such an alert allows the operator to accurately recognize the actual recommended operation direction and correct the operation appropriately.

Furthermore, after outputting recommended information, when the operation direction by the operator is different than the recommended operation direction and it is not a situation where an operation in the recommended operation direction is difficult, the recommended operation presentation system can provide other recommended operation information. With the configurations described above, the recommended operation presentation system can present various insertion operations in accordance with a situation and allows the operator to try to insert the insertion section deep into the depths of the object, even if adhesion, travel abnormality of the large intestine, or the like has occurred.

Furthermore, in order to proceed with the straightening of the insertion section 11 during releasing an N-loop or an α-loop in a stepwise manner, after determining that a recommended operation in accordance with specific recommended operation information has been completed, the recommended operation presentation system determines the presence or absence of further attention state information (reference attention state information) based on recommended operation information corresponding to the recommended operation information, and if there is further attention state information, the recommended operation presentation system calculates recommended operation information based on the further attention state information and presents the calculated information. With this configuration, the recommended operation presentation system can present a sequence of recommended operation information and lead completion of the insertion.

Hereinafter, the display of recommended operation information etc. on the display device 30 will be described.

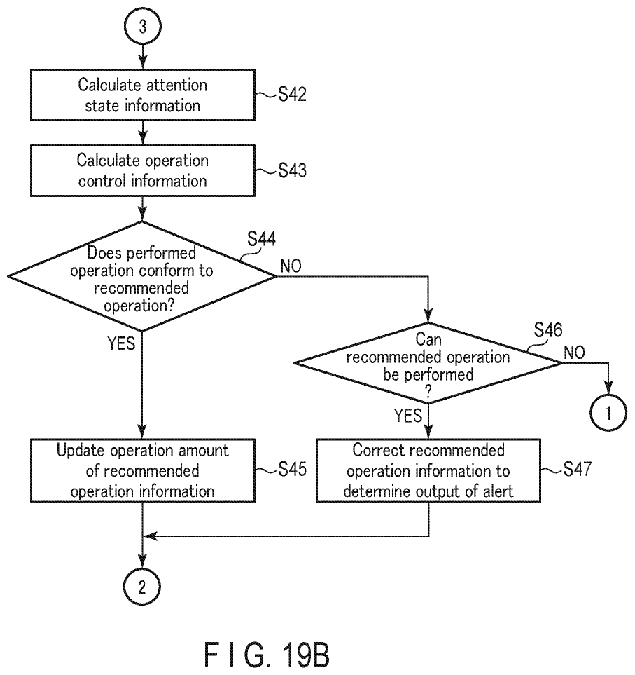

FIG. 9 is a diagram showing an example of a display screen 200 of the display device 30. An endoscope image 201 and an endoscope shape image 202 are displayed on the display screen 200. An endoscope shape image 202 includes an endoscope shape 203. Recommended operation information 210 is shown on the display screen 200. FIG. 9 shows, for example, a bar graph 211 indicating an insertion/extraction operation and an insertion/extraction amount, a pie graph 212 indicating a twisting operation and a twist amount, and four bar graphs 213 each having an arrow shape indicating a bending operation and a bending amount. The four bar graphs 213 are arranged one each at the top and bottom and left and right of the periphery of the endoscope image 201.

The bar graph 211 indicates an operation amount 214 with oblique lines within a predetermined range above a solid line at the center of the graph. This indicates, for example, a predetermined amount of a pushing operation as recommended operation information. The pie graph 212 shows an operation amount 214 with oblique lines within a predetermined angle range on the right side from a solid line in the circle. This indicates, for example, a predetermined amount of a counter-clockwise twisting operation as recommended operation information. The bar graph 213 on the right side of the endoscope image 201 indicates an operation amount 214 with oblique lines within a predetermined range from the left side within the arrow. This indicates, for example, a predetermined amount of right bending operation as recommended operation information.

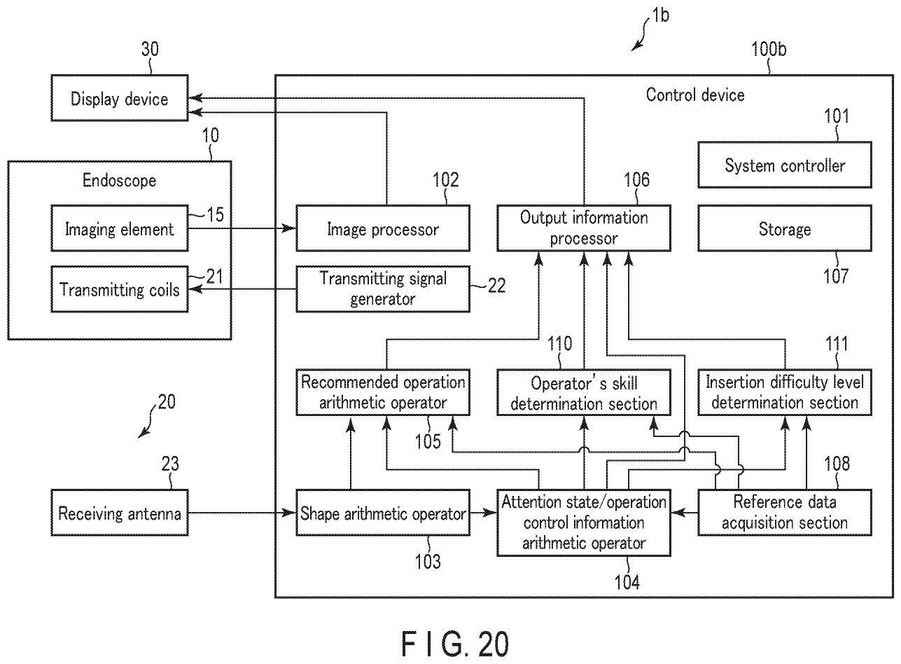

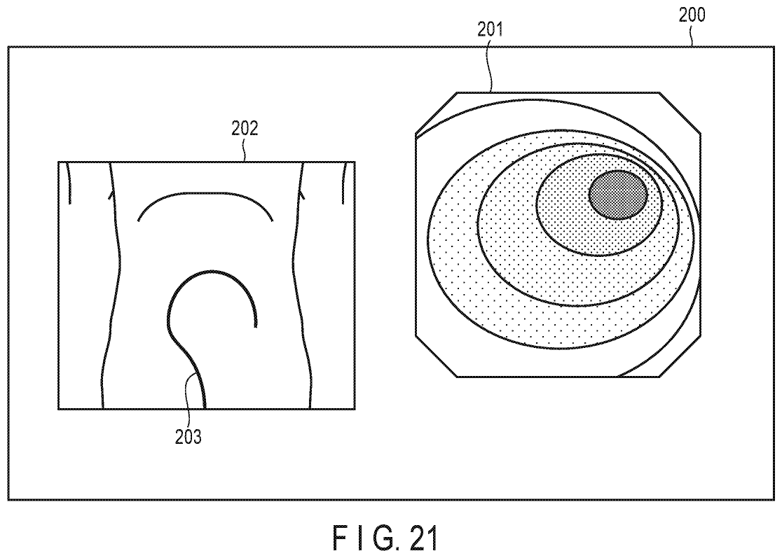

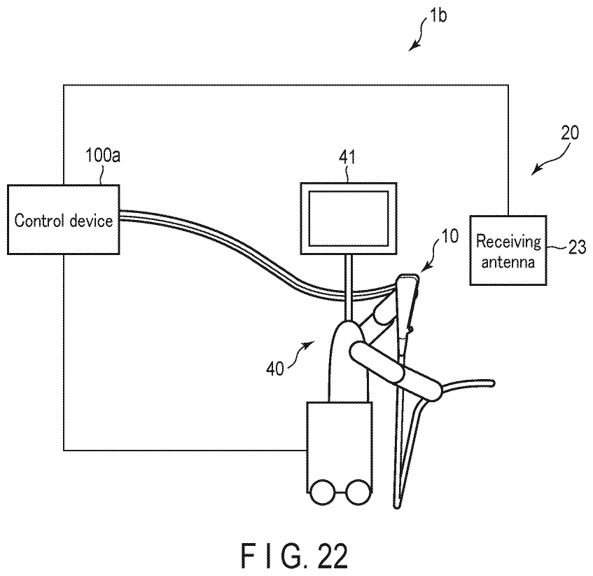

FIGS. 10A to 100 show examples of the display of the recommended operation information. The operation amounts 214 in a bar graph 211, a pie graph 212, and a bar graph 213 in the form of an arrow decrease as the operator performs the recommended operation as shown in FIGS. 10A to 100. Namely, the operation amount 214 represents the remaining operation amount with respect to the target shape. Such a display allows the operator to visually perceive the remaining operation amount.

FIGS. 11A to 11C show other examples of the display of the recommended operation information. A bar graph 211, a pie graph 212, and a bar graph 213 in the form of an arrow each indicate an operation amount 218 detected by various sensors capable of detecting a bending operation, a twisting operation or an insertion/extraction operation, and a recommended operation amount 219. In this case, the operator can perform the recommended operation until the detected operation amount 218 becomes the recommended operation amount 219.

Of course, the display of the recommended operation information 210 is not limited to a bar graph and a pie graph, but it suffices that the display shows the direction of an operation (the direction of a bending operation, twisting operation, or insertion/extraction operation) and the amount of the operation.

Referring again to FIG. 9, attention state information 215 may be displayed on the display screen 200. FIG. 9 presents an occurrence of an N-loop (generally ρ-character). This allows the operator to recognize, in addition to the endoscope shape image 202 including the endoscope shape 203, the occurrence of the N-loop.

The display screen 200 may display recommended operation conformity information 216 indicating whether or not the recommended operation direction and the operation direction by the operator conform to each other. In FIG. 9, out of “Operation OK” and “Not conform to recommended operation”, which are the recommended operation conformity information, the inside of a frame of “Operation OK” is shown by oblique lines. This indicates that the recommended operation direction and the operation direction by the operator conform to each other. This allows the operator to proceed through the insertion while checking that the operation performed by the operator matches with the recommended operation.

The display unit 200 may display a notification section 217 for notifying an operator that the recommended operation information 210 and attention state information 215 are displayed. In FIG. 9, the notification section 217 is placed on the lower left side of the endoscope image 201. The notification section 217 is preferably superimposed on the endoscope image 201 or placed near the endoscope image 201. This makes it easier for the operator to perceive that the recommended operation information 210 and the attention state information 215 is displayed even if the operator is paying close attention to the endoscope image 201.

For example, during N-loop release, there is a case where the straightening of an N-loop may be successful if the operation is performed slowly, but the straightening fails if the operation is preformed quickly; for example, there is a case where the insertion section 11 recedes from the large intestine. Also, there is a case where a plurality of operations, such as a pulling operation and a bending operation, are performed simultaneously. In this case, the amount of the bending operation with respect to the pulling operation is important.

Therefore, as shown in FIGS. 12A to 12C, a recommended operation speed 220 may be displayed as the recommended operation information 210 in addition to the detected operation amount 218 and the recommended operation amount 219. In other words, a recommended operation amount for each moment of operation is displayed as the recommended operation speed 220. In FIGS. 12A to 12C, the recommended operation speed 220 is displayed using a dotted line. The dotted line is displayed while being changed in the direction of a recommended operation amount with time from the start of the display (the graphs 211, 212, 213) of the recommended operation information 210.

As described above, the recommended operation information may include a recommended operation speed, which is a speed of a recommended insertion operation. The recommended operation speed is calculated by the recommended operation arithmetic operator 105, based on a change of time of at least one of the attention state information and the operation control information. The operator performs an operation such that a recommended operation speed conforms to a detected operation amount. With this configuration, a more detailed operation method can be presented, and thus the insertability of the insertion section 11 is improved.

As shown in FIG. 13, the recommended operation information 210 may be temporarily superimposed on the endoscope image 201 when the display is necessary. Since the operator usually pays close attention on the endoscope image 201, by superimposing the recommended operation information 210 on the endoscope image 201, the operator can be easily aware of the recommended operation information 201. Also, the operator does not need to look aside from the endoscope image 201. The attention state information 215 may be superimposed in addition to the recommended operation information 210. On the other hand, when the recommended operation information 210 and the attention state information 215 are superimposed on the endoscope image 201, the endoscope image 201 becomes invisible at the superimposed position, or it becomes difficult to view the endoscope image 201 even if the image is translucent. Therefore, information obtained from the endoscope image 201 is reduced. Therefore, the reduction in the amount of information obtained from the endoscope image 201 can be prevented by temporarily displaying the information in a superimposed manner when necessary. FIG. 13 shows a hidden state (left), a displayed state (center), and a hidden state (right) of the recommended operation information 210 from the left to the right, and these states are suitably switched by the operator.

The recommended operation information 210 or the attention state information may be displayed in a moving image as shown in FIG. 14. FIG. 14 shows an animation 221 of an operation in which the insertion section 11 is pulled by the operator's hand 222. The type of the moving image may be real-life, CG, or animation. The display of moving images allows the operator to visually understand the contents of a recommended operation and the contents of an attention state.

The recommended operation information and attention state information generated during a short period of time (e.g., one second) may be set to be displayed for a predetermined period of time longer than that of the short time (e.g., 5 seconds). For example, as shown in FIG. 15, if a loop is generated for 10 seconds, then the output information processor 106 outputs recommended operation information and attention state information for the loop for 10 seconds from the start to the end of the occurrence of the loop and causes the display device 30 to display the information. This period of time is sufficient for the operator to recognize the information. However, if a loop is generated only for a short time, for example, for about 1 second or less, there is a concern that the operator may overlook the display or may not be able to recognize the contents of the display, even though the operator can recognize that something is displayed. Therefore, for a loop generated for a short period of time, the display is started at the start of the occurrence of the loop, and the display is continued for a certain period of time, which seems to be necessary for the operator to recognize the contents, for example, for five seconds. This makes it easier for the operator to recognize the displayed contents.

On the other hand, it is also considered that recommended operation information and attention state information generated for a short period of time does not have to be recognized by the operator. Therefore, the display may be set so as not to display such information on the display screen. For example, as shown in FIG. 16, the output information processor 106 does not output recommended operation information and attention state information for a certain period of time (e.g., 1 second) after the occurrence of a loop. Then, if the recommended operation information and attention state information is continued, that is, if a loop continues after a certain period of time has passed, then the recommended operation information and the attention state information is output. On the other hand, if the loop does not continue after a certain period of time has passed, then the output information processor 106 does not output the attention state information and the recommended operation information. This allows not displaying information under conditions in which the information does not have to be recognized by the operator and is generated for a short period of time. In other words, the operator is more likely to easily recognize necessary information.

The output information processor 106 may change the display method in accordance with the attention state information and the recommended operation information. For example, the display may be set such that information necessary even for a short time is output for more than a certain period of time. When the information is unnecessary if displayed for a short period of time, the display may be set such that the information is not output.

As described above, recommended operation information is displayed for a time during which the operator easily recognizes the recommended operation information. This allows the operator to perform an operation while appropriately recognizing in which direction the operation should be performed and how much of the operation should be performed. Therefore, the operability is improved. The safety is also improved because an appropriate operation can be performed.

In the above descriptions, pushing, pulling, and twisting of the insertion section 11 are described as examples of the recommendation operation information. However, air supply, water supply, suction, change of the hardness degree of the insertion section 11, hand compression, and the like may be displayed as the recommended operation information in addition to the above examples.

An example of the recommended operation presentation system has been explained above, in which attention state information and operation control information is calculated based on the shape of the insertion section 11, and recommended operation information is determined. The recommended operation presentation system may be configured such that information obtained from the endoscope image 201 is further added to determine recommended operation information. For example, as shown in FIG. 17, a lumen region 223 including the position of the lumen is detected from endoscope image 201. In FIG. 17, the lumen of a detection result is displayed by being surrounded with a dashed frame. The frame may be changed between display/hidden when necessary. The recommended operation arithmetic operator 105 determines, as recommended information, an operation for directing the distal end of the insertion section 11 toward the direction of the lumen, based on positional information on the image of the detected lumen region 223. In this way, by adding information obtained from the endoscope image 201 and determining the recommended operation information, more detailed recommended operation information can be generated, and the accuracy of the recommended operation information can be improved.

Although an example of a recommended operation system configured to display recommended operation information as image information on the display device 30 is described above, the recommended operation information may be output by sound. In other words, recommended operation information output from the output information processor 106 may be at least one of visual information and auditory information. For example, when recommended operation information is started to be output, a sound is output at the interval of a period t. The period t becomes shorter as the current shape of the insertion section 11 gets closer to a target shape, and when the current shape has reached the target shape, a continuous sound is output. Not only a period but also a change of pitch may be used. For example, a higher-pitched sound is output as the current shape of the insertion section 11 gets closer to the target shape. Use of such a change of sound leads to an improvement in the operability because the change of sound allows the operator to recognize recommended operation information without viewing the recommended operation information on the monitor and to proceed to an insertion operation while focusing attention on observation of an endoscope image.

### Modification 1

In modification 1, a control device 100a of an endoscope system 1a includes an insertion state determination unit 109 in addition to the above-described sections, as shown in FIG. 18. Hereinafter, an example of the processing by the control device 100 of the endoscope system 1 as a recommended operation presentation system according to the modification 1 will be described schematically with reference to FIGS. 19A and 19B.

Steps S31 to S33 are similar to steps S11 to S13 shown in FIGS. 4A and 4B, respectively. In step S33, if it is determined that the attention state information does not apply to the reference attention state information, or if it is determined from the recommended operation necessity determination information that the recommended operation information is unnecessary even if the recommended operation information applies to the reference attention state information (step S33—NO), the processing proceeds to step S41. If it is determined that the attention state information applies to the reference attention state information, or if it is determined from the recommended operation necessity determination information that the recommended operation information is necessary even if the attention state information does not apply to the reference attention state information (step S33—YES), the processing proceeds to step S34.

In step S34, the insertion state determination unit 109 determines whether or not the insertion is in a state of going through difficulties. For example, in a state where after recommended operation information is output, the processing does not proceed to the next recommended operation but continues for a certain period of time, or a case where even if the processing proceeds to the next recommended operation, the processing returns to the previous processing again has occurred a certain number of times or more, the insertion is determined to be in a state of going through difficulties. Also, the insertion may be determined to be in a state of going through difficulties if the attention state is not in the recommended operation reference data. In this case, it is desirable that an attention state generated in a typical insertion be adequately stored as recommended operation reference data in the storage 107 or another storage device. Also, the lumen is detected from an endoscope image by an image recognition technique, and if the lumen is not detected for a certain period of time, the insertion may be determined to be in a state of going through difficulties.

When the insertion is determined to be in a state of going through difficulties (step S34—YES), the processing proceeds to step S48. As one strategy for addressing a situation where the insertion is in a state of going through difficulties, an operation of pulling the insertion section 11 once may be sometimes used. This is because the insertion sometimes proceeds by restoring the previous situation (i.e., the situation prior to the situation where the insertion enters a state of going through difficulties) and repeating the insertion operation. Also, if the lumen is out of the operator's sight, the lumen is highly likely to be able to be found by pulling the insertion section 11. Therefore, in step S48, the recommended operation arithmetic operator 105 changes the recommended operation information to a test operation (e.g., a pulling operation, a clockwise twisting operation, and a counter-clockwise twisting operation) for changing the insertion state. The operation may be an operation other than the above-mentioned operations. The operation direction relating to the test operation may also be included in the recommended operation reference data. Through such a test operation, it becomes possible to find a lumen that has been out of the operator's sight, and a lumen that is entangled in the insertion section 11 is loosened, and thereby the insertion can be performed easily.

In this way, even in a state where the insertion enters a state of going through difficulties and recommended operation information cannot be issued, the insertion state can be changed by determining, by the insertion state determination section, the state where the insertion is going through difficulties, and changing the recommended operation information to a trial operation (i.e., a practice to get better). This enhances a potential of making recommended operation information able to be output or making recommended operation information be in a state of easy insertion of the insertion section 11 without the need for recommended operation information.

On the other hand, when it is determined that the situation is not a state where the insertion is in a state of going through difficulties (step S34—NO), the processing proceeds to step S35. The contents of steps S35 through S47 are respectively similar to those of steps S14 through S26 in FIGS. 4A and 4B.

### Modification 2

In modification 2, the control device 100b of the endoscope system 1b includes an operator's skill determination unit 110 and an insertion difficulty level determination unit 111 in addition to the above-mentioned section, as shown in FIG. 20.

[Operator's Skill Determination Section]

The operator's skill determination unit 110 determines the level of an operator's insertion operation skill (operator's level) for the endoscope 10 based on the attention state information generated during the insertion. The operator's skill determination unit 110 comprehensively determines, for example, the time taken for straightening an N-loop, the number of times the operator failed to straighten a loop from the N-character shape and re-started the operation again from the generally ρ-character, thereby determining the operator's level. The number of times a buckling state occurred in a portion of the insertion section 11 and the number of times the intestinal extension occurred may be added to elements of the determination. In other words, the operator's level is determined based on at least one of the type of attention state information generated during a series of insertion operations by an operator, the number of occurrence times, generated time, generated location, and the state of an object. Also, the time required for the occurrence of the above-mentioned attention state, the time required to complete the insertion, and the time required to release the loop or the like may be stored in the storage 107 or other storage device as the user's past insertion operation history data, and the operator's skill determination unit 110 may determine with reference to the data.

[Insertion Difficulty Level Determination Section]

The insertion difficulty level determination section 111 determines, based on the state information generated during insertion, whether or not the large intestine as an object is in a state where it is difficult to insert the insertion section, for example. In other words, the insertion difficulty level is determined based on at least one of the type of attention state information generated during a series of insertion operations by an operator, the number of occurrence times, generated time, generated location, and the state of an object. For example, if a double α-loop is generated, it can be inferred that the intestine adheres, so the large intestine is determined to be in a state where it is difficult to insert the insertion section 11 into the large intestine. Additionally, the insertion time, surgical history (e.g., intestinal adhesions resulting from an operation in the patient's surgical history of operations), CT data, etc. may be stored in the storage 107 or other storage device as historical data of the patient's past insertion operation history data, and the insertion difficulty level determination section 111 may refer to these pieces of data to determine the insertion difficulty level.

The output information processor 106 changes the output content of the recommended operation information based on a result determined by the operator's skill determination unit 110 and a result determined by the insertion difficulty level determination section 111. For example, if it is determined that the operator's skill level is high and the difficulty level of the insertion object is low, then the output information processor 106 determines that no recommended operation information is necessary, and does not output recommended operation information, and as shown in FIG. 21, only an endoscope image 201 and an endoscope shape image 202 are displayed on a display screen 200. In response to the result determined by the operator's skill determination unit 110 and the result determined by the insertion difficulty level determination section 111, only some of the recommended operation information may be output appropriately. Such output/display makes it easier for the operator to obtain necessary information.

### Modification 3

In modification 3, recommended operation information is output as a control signal of an automatic insertion device 40 to the automatic insertion device 40, which automatically inserts the endoscope 10 or partially automatically inserts the endoscope 22, as shown in FIG. 22. The automatic insertion device 40 may include a display unit 41, and the display unit 41 may substitute for the display device 30. Even in such an insertion operation by the automatic insertion device 40, the processing by the recommended presentation system as described above can be performed, and similar effects can be obtained.

Note that the invention of the present application is not limited to the above embodiments and can be variously modified in a range not departing from the gist in an implementation stage. In addition, the embodiments may be implemented in appropriate combinations as much as possible, and in that case, combined effects are obtained. Furthermore, the above embodiments include inventions at various stages, and various inventions can be extracted by appropriate combinations of plural constituent elements disclosed.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A recommended operation presentation system, comprising:
an insertion instrument including an insertion section to be inserted into an insertion object;
an insertion section shape detector configured to detect a shape of at least part of the insertion section as shape information;
an attention state/operation control information arithmetic operator configured to calculate operation control information that is information changing in association with an operation of the insertion instrument, based on the shape information and target state information, the target state information being set based on the shape information and preliminarily prepared recommendation operation reference data;
a recommended operation arithmetic operator configured to calculate recommended operation information including a recommended operation direction that is an operation direction of a recommended insertion operation and a recommended operation amount that is an operation amount of the recommended insertion operation, based on at least the operation control information; and
an output information processor configured to output information including the recommended operation information.

2. The recommended operation presentation system according to claim 1, further comprising:
a reference data acquisition section configured to acquire the recommended operation reference data, wherein
the recommended operation reference data includes reference attention state information relating to a predetermined state of the insertion section, and an operation direction and the target state information that are preliminarily set so as to correspond to the reference attention state information,
the attention state/operation control information arithmetic operator is configured to
calculate attention state information relating to the predetermined state of the insertion section based on the shape information, and
calculate the operation control information based on at least the reference attention state information corresponding to the attention state information and the target state information; and
the recommended operation arithmetic operator is configured to
calculate the recommended operation direction based on the attention state information and the operation direction, and
calculate the recommended operation amount based on at least the operation control information.

3. The recommended operation presentation system according to claim 1, wherein
the recommended operation reference data includes options of pieces of target state information in accordance with the shape information, and
the attention state/operation control information arithmetic operator is configured to select target state information in accordance with the shape information, from among the options of the pieces of target state information that are preliminarily prepared.

4. The recommended operation presentation system according to claim 2, wherein
the recommended operation arithmetic operator is configured to determine, after outputting the recommended operation information, from current shape information detected by the insertion section shape detector, whether or not a recommended operation in accordance with the recommended operation information is completed, and
when the recommended operation is determined to not have been completed:
the attention state/operation control information arithmetic operator is configured to calculate the attention state information and the operation control information based on the current shape information again; and
the recommended operation arithmetic operator is configured to calculate the recommended operation information again, and the output information processor is configured to output information including the recommended operation information again.

5. The recommended operation presentation system according to claim 2, wherein
the recommended operation information includes a recommended operation speed that is a speed of a recommended insertion operation, and
the recommended operation arithmetic operator is configured to calculate the recommended operation speed based on at least one of the attention state information and the operation control information.

6. The recommended operation presentation system according to claim 2, wherein
recommended operation necessity determination information is preliminarily set so as to correspond to the reference attention state information, and
the recommended operation arithmetic operator is configured to:
determine the necessity of the recommended operation information based on the attention state information and the corresponding recommended operation necessity determination information; and
when determining that the recommended operation information is necessary, calculate the recommended operation information.

7. The recommended operation presentation system according to claim 1, wherein
the recommended operation arithmetic operator is configured to:
determine whether or not a recommended operation in accordance with the recommended operation information has been completed;
when determining that the recommended operation has not been completed, determine whether or not the recommended operation can be performed; and
when determining that the recommended operation cannot be performed, calculate recommended operation information including a recommended operation direction relating to a trial operation different from the recommended operation that has not been completed.

8. The recommended operation presentation system according to claim 7, wherein
the trial operation is at least one of a pulling operation, a counter-clockwise twisting operation, and a clockwise twisting operation of the insertion section.

9. The recommended operation presentation system according to claim 7, wherein
the output information processor is configured to output a visual or auditory alert for an operation direction when determining that the recommended operation can be performed.

10. The recommended operation presentation system according to claim 2, wherein
the recommended operation arithmetic operator is configured to:
determine whether or not a recommended operation in accordance with the recommended operation information is determined to have been completed; and
when determining that the recommended operation has been completed, further determine the necessity of a further attention state information calculation based on recommended operation continuation information preliminarily set so as to correspond to the attention state information.

11. The recommended operation presentation system according to claim 2, wherein
the attention state information includes a type and a size of a loop formed in the insertion section,
the target state information is target shape information including the type and the size of the loop, and
the operation control information is information based on a difference between a shape relating to the shape information and a target shape relating to the target shape information.

12. The recommended operation presentation system according to claim 11, wherein
the attention state/operation control information arithmetic operator is configured to
calculate, as the attention state information, first attention state information that is a generally ρ-character shape having a predetermined size or larger, based on first shape information detected by the insertion section shape detector, and
calculate, as the operation control information, first operation control information that is a difference in size between the generally ρ-character shape relating to the first attention state information and a generally ρ-character shape having a size smaller than the predetermined size relating to the target shape information; and
the recommended operation arithmetic operator is configured to calculate, as the recommended operation information, first recommended operation information including a first recommended operation direction that is a pulling direction, and a first recommended operation amount based on the first operation control information,
after the first attention state information, the first operation control information, and the first recommended operation information are calculated:
the attention state/operation control information arithmetic operator is configured to
calculate, as the attention state information, second attention state information that is a generally ρ-character or N-character shape having a size smaller than the predetermined size, based on second shape information detected by the insertion section shape detector, and
calculate, as the operation control information, second operation control information that is a difference in angle of coordinates between the generally ρ-character or N-character shape relating to the second attention state information and an N-character shape relating to the target shape information; and
the recommended operation arithmetic operator is configured to calculate, as the recommended operation information, second recommended operation information including a second recommended operation direction that is a rotation direction, and a second recommended operation amount based on the second operation control information, and
after the second attention state information, the second operation control information, and the second recommended operation information are calculated:
the attention state/operation control information arithmetic operator is configured to
calculate, as the attention state information, third attention state information that is an N-character shape, based on third shape information detected by the insertion section shape detector, and
calculate, as the operation control information, third operation control information that is a sum of differences in coordinates between the N-character shape relating to the third attention state information and a generally straight line shape relating to the target shape information; and
the recommended operation arithmetic operator is configured to calculate, as the recommended operation information, third recommended operation information including a third recommended operation direction that is a pulling direction, and a third recommended operation amount based on the third operation control information.

13. The recommended operation presentation system according to claim 11, wherein
the attention state/operation control information arithmetic operator is configured to
calculate, as the attention state information, second attention state information that is a generally ρ-character or N-character shape having a size smaller than a predetermined size, based on second shape information detected by the insertion section shape detector, and
calculate, as the operation control information, second operation control information that is a difference in angle of coordinates between the generally ρ-character or N-character shape relating to the second attention state information and an N-character shape relating to the target shape information; and
the recommended operation arithmetic operator is configured to calculate, as the recommended operation information, second recommended operation information including a second recommended operation direction that is a rotation direction, and a second recommended operation amount based on the second operation control information, and
after the second attention state information, the second operation control information, and the second recommended operation information are calculated:
the attention state/operation control information arithmetic operator is configured to
calculate, as the attention state information, third attention state information that is an N-character shape, based on third shape information detected by the insertion section shape detector, and
calculate, as the operation control information, third operation control information that is a sum of differences in coordinates between the N-character shape relating to the third attention state information and a generally straight line shape relating to the target shape information; and
the recommended operation arithmetic operator is configured to calculate, as the recommended operation information, third recommended operation information including a third recommended operation direction that is a pulling direction, and a third recommended operation amount based on the third operation control information.

14. The recommended operation presentation system according to claim 11, wherein
the attention state/operation control information arithmetic operator is configured to
calculate, as the attention state information, third attention state information that is an N-character shape, based on third shape information detected by the insertion section shape detector, and
calculate, as the operation control information, third operation control information that is a sum of differences in coordinates between an N-character shape relating to the third attention state information and a generally straight line shape relating to the target shape information; and
the recommended operation arithmetic operator is configured to calculate, as the recommended operation information, third recommended operation information including a third recommended operation direction as a pulling direction, and a third recommended operation amount based on the third operation control information.

15. The recommended operation presentation system according to claim 11, wherein
the attention state/operation control information arithmetic operator is configured to
calculate, as the attention state information, fourth attention state information that is an α-loop or left-right reversed α-loop shape having a predetermined size or larger, based on fourth shape information detected by the insertion section shape detector, and
calculate, as the operation control information, fourth operation control information that is a difference in size between the α-loop or left-right reversed α-loop shape relating to the fourth attention state information and an α-loop or left-right reversed α-loop shape having a size smaller than the predetermined size relating to the target shape information; and
the recommended operation arithmetic operator is configured to calculate, as the recommended operation information, fourth recommended operation information including a fourth recommended operation direction that is a pulling direction, and a fourth recommended operation amount based on the fourth operation control information, and
after the fourth attention state information, the fourth operation control information, and the fourth recommended operation information are calculated:
the attention state/operation control information arithmetic operator is configured to
calculate, as the attention state information, fifth attention state information that is an α-loop or left-right reversed α-loop shape based on fifth shape information detected by the insertion part shape detector and
calculate fifth operation control information that is a sum of differences in coordinates between the α-loop or left-right reversed α-loop shape relating to the fifth shape information and a generally straight line shape relating to the target shape information; and
the recommended operation arithmetic operator is configured to calculate fifth recommended operation information including a fifth recommended operation direction that is a rotation direction, and a fifth recommended operation amount based on the fifth operation control information.

16. The recommended operation presentation system according to claim 11, wherein
the attention state/operation control information arithmetic operator is configured to
calculate, as the attention state information, fifth attention state information that is an α-loop or left-right reversed α-loop shape having a size smaller than a predetermined size, based on fifth shape information detected by the insertion section shape detector, and
calculate, as the operation control information, fifth operation control information that is a sum of differences in coordinates between the α-loop or left-right reversed α-loop shape relating to the fifth attention state information and a generally straight line shape relating to the target shape information; and
the recommended operation arithmetic operator is configured to calculate, as the recommended operation information, fifth recommended operation information including a fifth recommended operation direction that is a rotation direction, and a fifth recommended operation amount based on the fifth operation control information.

17. The recommended operation presentation system according to claim 16, wherein
when the fifth attention state information is an α-loop in which an insertion section distal end side is present on an upper side at an intersecting portion of the insertion section, or a left-right reversed α-loop in which the insertion section distal end side is present on a lower side at the intersection portion of the insertion section, the rotation direction is a clockwise twisting direction, and
when the fifth attention state information is an α-loop in which the insertion section distal end side is present on the lower side at the intersection portion of the insertion section, or a left-right reversed α-loop in which the insertion section distal end side is present on the upper side at the intersection portion of the insertion section, the rotation direction is a leftward twist direction.

18. The recommended operation presentation system according to claim 2, wherein
the attention state information is at least one of:
whether or not the insertion section forms a loop with an α-loop, a top-bottom reversed α-loop, a left-right reversed α-loop, a top-bottom and left-right reversed α-loop, an N-character shape or a generally ρ-character shape, and a size of the loop including an area, a circumference, and a width;
a movement amount of the insertion section distal end; and
a proportion of the movement amount of the insertion section distal end with respect to an insertion amount.

19. The recommended operation presentation system according to claim 2, further comprising:
a storage configured to store the recommended operation reference data, wherein
the reference data acquisition section is configured to acquire the recommended operation reference data from the storage.

20. The recommended operation presentation system according to claim 1, wherein
the operation control information is at least one of an insertion/extraction operation amount of the insertion section, a rotational operation amount of the insertion section, an angle operation amount of the insertion section, air supply, water supply, suction, a body posture of a patient, a hand compression, and a rigidity of the insertion section.

21. The recommended operation presentation system according to claim 1, wherein
the output information processor is configured to cause the recommended operation information to be displayed with at least one of a bar graph and a pie graph.

22. The recommended operation presentation system according to claim 2, further comprising:
an operator's level determination section configured to determine, based on the attention state information, an operator's level that is an operation skill level of an operator of the insertion instrument, wherein
the output information processor is configured to change an output content based on the operator's level.

23. The recommended operation presentation system according to claim 22, wherein
the operator's level is determined based on at least one of a type, the number of times, occurrence time, and an occurrence place of the attention state information occurring during a series of insertion operations by the operator, and a state of the insertion object, or based on past insertion history data preliminarily set for the operator.

24. The recommended operation presentation system according to claim 2, further comprising:
an insertion difficulty level determination section configured to determine an insertion difficulty level of an insertion object based on the attention state information, wherein
the output information processor is configured to change an output content based on the insertion difficulty level.

25. The recommended operation presentation system according to claim 24, wherein
the insertion difficulty level is determined based on at least one of a type, the number of times, occurrence time, and an occurrence place of attention state information occurring during a series of insertion operations by the operator, and a state of the insertion object, or based on past insertion history data preliminarily set for the operator.

26. The recommended operation presentation system according to claim 2, wherein
the insertion section includes an imager, and
the output information processor is configured to cause at least one of the attention state information and the recommended operation information to be displayed or temporarily displayed with being superimposed on an image taken by the imager.

27. The recommended operation presentation system according to claim 2, wherein
the output information processor is configured to cause at least one of the attention state information and the recommended operation information to be displayed in a video.

28. The recommended operation presentation system according to claim 1, wherein
the output information processor is configured to cause the recommended operation information to be displayed for a preliminarily set time or longer.

29. The recommended operation presentation system according to claim 2, wherein
the output information processor is configured to cause the recommended operation information not to be displayed if an occurrence time of an attention state of an object for which the recommended operation information is to be output is equal to or shorter than a preliminarily set time.

30. The recommended operation presentation system according to claim 29, wherein
the output information processor is configured not to output the recommended operation information for a predetermined set time after an occurrence of the attention state relating to the attention state information that has become a cause of outputting the recommended operation information and to output the recommended operation information if the attention state continues after the predetermined set time has elapsed.

31. The recommended operation presentation system according to claim 2, wherein
the insertion section comprises an imager, and
the recommended operation arithmetic operator is configured to determine the recommended operation information based on image information taken by the imager together with the attention state information.

32. The recommended operation presentation system according to claim 1, wherein
the insertion instrument is an endoscope, and the insertion section is a flexible tube section of the endoscope.

33. A recommended operation presentation control device comprising:
an attention state/operation control information arithmetic operator configured to calculate operation control information that is information changing in association with an operation of an insertion instrument including an insertion section configured to be inserted into an insertion object, based on shape information and target state information, the shape information relating to a shape of at least part of the insertion section, the target state information being set based on the shape information and preliminarily prepared recommendation operation reference data;
a recommended operation arithmetic operator configured to calculate recommended operation information including a recommended operation direction that is an insertion direction of a recommended insertion operation and a recommended operation amount that is an operation amount of the recommended insertion operation, based on at least the operation control information; and
an output information processor configured to output information including the recommended operation information.

34. A recommended operation presentation control method comprising:
calculating operation control information that is information changing in association with an operation of an insertion instrument including an insertion section configured to be inserted into an insertion object, based on shape information and target state information, the shape information relating to a shape of at least part of the insertion section, the target state information being set based on the shape information and preliminarily prepared recommendation operation reference data;
calculating recommended operation information including a recommended operation direction that is an insertion direction of a recommended insertion operation and a recommended operation amount that is an operation amount of the recommended insertion operation, based on at least the operation control information; and
outputting information including the recommended operation information.
